SEQUENCE LISTING (18 sequences)

SEQ ID NO: 1 - protein, 11 aa, synthetic construct
QQKFKFKFKQQ

SEQ ID NO: 2 - protein, 11 aa, synthetic construct
EQEFEFEFEQE

SEQ ID NO: 3 - protein, 11 aa, synthetic construct
KQKFKFKFKQK

SEQ ID NO: 4 - protein, 11 aa, synthetic construct
DQDFDFDFDQD

SEQ ID NO: 5 - protein, 11 aa, synthetic construct
KQQFKFKFKQQ

SEQ ID NO: 6 - protein, 11 aa, synthetic construct
QQKFQFQFKQQ

SEQ ID NO: 7 - protein, 11 aa, synthetic construct
QQEFEFEFEQQ

SEQ ID NO: 8 - protein, 11 aa, synthetic construct
KQQFKFKFQQK

SEQ ID NO: 9 - protein, 11 aa, synthetic construct
QQEFQFQFEQQ

SEQ ID NO: 10 - protein, 20 aa, synthetic construct
QQKFKFKFKQQGSGGSGLLR

SEQ ID NO: 11 - protein, 20 aa, synthetic construct
KQKFKFKFKQKGSGGSGLLR

SEQ ID NO: 12 - protein, 20 aa, synthetic construct
EQEFEFEFEQEGSGGSGLLR

SEQ ID NO: 13 - protein, 20 aa, synthetic construct
DQDFDFDFDQDGSGGSGLLR

SEQ ID NO: 14 - protein, 19 aa, synthetic construct
QQKFKFKFKQQGSGGSGLL

SEQ ID NO: 15 - protein, 19 aa, synthetic construct
EQEFEFEFEQEGSGGSGLL

SEQ ID NO: 16 - protein, 19 aa, synthetic construct
KQKFKFKFKQKGSGGSGLL

SEQ ID NO: 17 - protein, 19 aa, synthetic construct
DQDFDFDFDQDGSGGSGLL

SEQ ID NO: 18 - protein, 6 aa, synthetic construct
  SITE 1: note = isovaline
  SITE 4: note = X is statine
  SITE 6: note = X is statine
VVVXAX

(19) United States
(12) Patent Application Publication
(10) Pub. No.: US 2026/0347396 A1
(43) Pub. Date: Nov. 5, 2026

(54) COMPOSITIONS AND METHODS FOR SUPPRESSION OF ANTI-DRUG ANTIBODIES AND DELIVERY OF PROTEIN DRUGS

(71) Applicant: University of Florida Research Foundation, Incorporated, Gainesville, FL (US)

(72) Inventors: Gregory Allan Hudalla, Gainesville, FL (US); Lucas Melgar, Gainesville, FL (US)

(73) Assignee: University of Florida Research Foundation, Incorporated, Gainesville, FL (US)

(21) Appl. No.: 19/643,994

(22) Filed: Apr. 10, 2026

Related U.S. Application Data
(60) Provisional application No. 63/786,528, filed on Apr. 10, 2025.

(51) Int. Cl. C07K 5/083; A61K 38/00; C07K 7/06; C12N 9/16

(52) U.S. Cl. CPC C07K 5/0808; C07K 7/06; C12N 9/16; C12Y 301/03005; A61K 38/00; C07K 2319/00

(57) ABSTRACT

Aspects of the application relate to a protein or peptide comprising a charge-complementary peptide and a protease inhibitor (e.g., leupeptin). In some embodiments, the protein or peptide further comprises a therapeutic protein or therapeutic peptide, such as an enzyme (e.g., adenosine synthase A). Aspects of the application also relate to compositions comprising a plurality of positive peptides and a plurality of negative peptides, wherein the positive peptides and negative peptides are co-assembling peptides and wherein the positive peptides, the negative peptides, or both are each fused to a protease inhibitor (e.g., leupeptin). The application also provides methods of administering the proteins or compositions to a subject. In some embodiments, the proteins are administered to a subject repeatedly.

### RELATED APPLICATIONS

This application claims the benefit under 35 U.S.C. § 119(e) of U.S. Provisional Application No. 63/786,528, filed on Apr. 10, 2025, the disclosure of which is incorporated herein by reference in its entirety.

### REFERENCE TO AN ELECTRONIC SEQUENCE LISTING

The contents of the electronic sequence listing (U119770260US01-SEQ-JXV.xml; Size: 17,893 bytes; and Date of Creation: Apr. 9, 2026) are herein incorporated by reference in their entirety.

### BACKGROUND

Administration of a peptide or protein (e.g., a therapeutic peptide or therapeutic protein) to a subject may result in production of antibodies that reduce or eliminate the activity of the peptide or protein in the subject, especially under circumstances of repeated administration or local depots.

### SUMMARY

Peptides and proteins (e.g., therapeutic peptides or therapeutic proteins, such as enzymes) are an increasingly valuable tool to treat a myriad of diseases; however, the use of many peptides and proteins is compromised by the body's production of anti-drug antibodies (ADAs), especially under circumstances of repeated administration or local depots. Accordingly, provided herein are peptides, proteins, and compositions which reduce undesired humoral immune responses associated with peptides and proteins (e.g., therapeutic peptides or therapeutic proteins, such as enzymes).

In some aspects, the disclosure provides a peptide or protein comprising a charge-complementary peptide and a protease inhibitor. In some embodiments, the charge-complementary peptide comprises a positive peptide, optionally wherein the positive peptide comprises SEQ ID NO: 1 or 3. In some embodiments, the charge-complementary peptide comprises a negative peptide, optionally wherein the negative peptide comprises SEQ ID NO: 2 or 4. In some embodiments, the protease inhibitor comprises a leupeptin or a modified form thereof. In some embodiments, the protease inhibitor comprises a leupeptin having the sequence LLR. In some embodiments, the leupeptin is in an oxidized form. In some embodiments, the leupeptin is in a reduced form. In some embodiments, the protease inhibitor comprises a Pepstatin A or a modified form thereof. In some embodiments, the protease inhibitor comprises an amino acid sequence of {Iso-Val} {Val} {Val} {Sta} {Ala} {Sta}, wherein “Iso-Val” is isovaline and “Sta” is statine (SEQ ID NO: 18).

In some embodiments, any one of the peptides or proteins described herein further comprises a therapeutic protein or therapeutic peptide, optionally wherein the therapeutic protein or therapeutic peptide is an enzyme, further optionally wherein the enzyme is selected from: indoleamine 2,3-dioxygenase, adenosine synthase A, luciferase, or a combination thereof. In some embodiments, the enzyme is adenosine synthase A. In some embodiments, the therapeutic protein or therapeutic peptide is linked to the peptide or protein via a linker, optionally wherein the linker is a glycine-serine rich linker.

Some aspects of the disclosure provide a composition comprising a plurality of positive peptides, each fused to a protease inhibitor and a plurality of negative peptides, wherein the positive peptides and negative peptides are co-assembling peptides.

Some aspects of the disclosure provide a composition comprising a plurality of negative peptides, each fused to a protease inhibitor and a plurality of positive peptides, wherein the positive peptides and negative peptides are co-assembling peptides.

Some aspects of the disclosure provide a composition comprising a plurality of positive peptides, each fused to a protease inhibitor and a plurality of negative peptides, each fused to a protease inhibitor, wherein the positive peptides and negative peptides are co-assembling peptides.

In some embodiments, the positive peptide comprises SEQ ID NO: 1 or 3. In some embodiments, the negative peptide comprises SEQ ID NO: 2 or 4. In some embodiments, the protease inhibitor comprises a leupeptin or a modified form thereof. In some embodiments, the protease inhibitor comprises a leupeptin having the sequence LLR. In some embodiments, the leupeptin is in an oxidized form. In some embodiments, the leupeptin is in a reduced form. In some embodiments, the protease inhibitor comprises a Pepstatin A or a modified form thereof. In some embodiments, the protease inhibitor comprises an amino acid sequence of {Iso-Val} {Val} {Val} {Sta} {Ala} {Sta}, wherein “Iso-Val” is isovaline and “Sta” is statine (SEQ ID NO: 18).

In some embodiments, the plurality of positive peptides each further comprise a therapeutic protein or therapeutic peptide. In some embodiments, the plurality of negative peptides each further comprise a therapeutic protein or therapeutic peptide. In some embodiments, the plurality of positive peptides and the plurality of negative peptides each further comprise a therapeutic protein or therapeutic peptide.

In some embodiments, the therapeutic protein or therapeutic peptide is linked to the positive peptide or to the negative peptide via a linker. In some embodiments, the linker is a glycine-serine rich linker. In some embodiments, the therapeutic protein or therapeutic peptide is an enzyme, optionally wherein the enzyme is selected from: indoleamine 2,3-dioxygenase, adenosine synthase A, luciferase, or a combination thereof. In some embodiments, the enzyme is adenosine synthase A.

Some aspects or embodiments include a method comprising administering any one of the peptides or proteins or compositions disclosed herein to a subject, optionally wherein the subject is a human subject. In some embodiments, the method further comprises administering the peptide or the protein or the composition to subject one, two, three, or four more times. In some embodiments, the method further comprises administering the therapeutic protein or the therapeutic peptide to the subject repeatedly following administration of the peptide or protein or composition.

In some embodiments, the protein, peptide, or composition is administered subcutaneously, intramuscularly, intravenously, intrathecally, intra-articularly, intra-ocularly, sub-gingivally, by inhalation, oral intravascularly, or combination thereof.

In some embodiments, the administration results in a reduced level of anti-drug antibodies relative to that of a second subject administered the therapeutic protein or the therapeutic peptide without the peptide, protein, or composition.

Some aspects or embodiments include a cell or cell population comprising any one of the peptides or proteins disclosed herein or a nucleic acid encoding any one of the peptides or proteins.

Some aspects or embodiments include a nucleic acid encoding any one of the peptides or proteins disclosed herein.

### BRIEF DESCRIPTION OF DRAWINGS

FIGS. 1A-1C show non-limiting embodiments of co-assembly tags based on charge complementarity (CATCH) peptides. FIG. 1A shows a CATCH hydrogel comprising oppositely charged peptides that, when combined, co-assemble into a nanofiber-based hydrogel (left panel) and the use of CATCH peptides for whole protein functionalization (right panel). FIG. 1B shows two examples of CATCH peptides. The sequence of the 4K+ peptide is SEQ ID NO: 1; the sequence of the 6E− peptide is SEQ ID NO: 2; the sequence of the 6K+ peptide is SEQ ID NO: 3; and the sequence of the 6D-peptide is SEQ ID NO: 4. FIG. 1C shows a non-limiting process for generating CATCH fusion proteins comprising CATCH proteins and an enzyme (e.g., AdsA, adenosine synthase A).

FIG. 2 provides examples of different CATCH fusion proteins comprising oxidized leupeptin (COOH) or reduced leupeptin (CH₂OH). Oxidation and reduction are relative to the natural aldehyde. The sequences, from top to bottom, correspond to SEQ ID NOs: 10-17.

FIG. 3 shows the relative IgG levels (anti-AdsA antibodies) in mice following administration of one or two full doses (100% leupeptin) of CATCH (4K+/6E−)-Leu (oxidized) hydrogel (initial administration and booster) at different time points. Note that mice were injected with the initial dose on day 0 and a booster dose on day 28. N=4; mean±standard deviation shown.

FIG. 4 shows the relative IgG levels (anti-AdsA antibodies) in mice following administration of one or two doses (1 mM) of CATCH (4K+/6E−)-Leu (oxidized) hydrogel (initial administration and booster) on days 21, 35, and 49. Note that mice were injected with the initial dose on day 0 and a booster dose on day 28. Mean±standard deviation shown.

FIG. 5 shows the relative IgG levels (anti-AdsA antibodies) in mice following administration of one or two doses (1 mM) of CATCH (6K+/6D−)-Leu (oxidized) hydrogel (initial administration and booster) on days 21, 35, and 49. Note that mice were injected with the initial dose on day 0 and a booster dose on day 28. Mean±standard deviation shown.

FIG. 6 shows the relative IgG levels (anti-AdsA antibodies) in mice following administration of one or two half doses (50%) of CATCH (4K+/6E−)-Leu hydrogel (initial administration and booster) at different time points. Note that mice were injected with the initial dose on day 0 and a booster dose on day 28. N=4; mean #standard deviation shown.

FIG. 7 shows the relative IgG levels (anti-AdsA antibodies) in mice following administration of one or two doses (1 mM) of CATCH (4K+/6E−)-Leu (reduced) hydrogel (initial administration and booster) on days 21, 35, and 49. Note that mice were injected with the initial dose on day 0 and a booster dose on day 28. Mean±standard deviation shown.

FIG. 8 shows the relative IgG levels (anti-AdsA antibodies) in mice following administration of one or two doses (1 mM) of CATCH (6K+/6D−)-Leu (reduced) hydrogel (initial administration and booster) on days 21, 35, and 49. Note that mice were injected with the initial dose on day 0 and a booster dose on day 28. Mean±standard deviation shown.

FIGS. 9A-9C shows the effects of different concentrations of different CATCH-leupeptin fusion proteins on the IgG levels (anti-AdsA antibodies) in mice on day 35 (FIG. 9A), and day 49 (FIG. 9B). The groups, from left to right, are Oxi-4K6E/CATCH (4K+/6E−)-Leu_V1), Oxi-6K6D/CATCH (6K+/6D−)-Leu_V1, Red-4K6E/CATCH (4K+/6E−)-Leu_V2, and Red-6K6D/CATCH (6K+/6D−)-Leu_V2 (“oxi” and “V1” represent the oxidized form of leupeptin and “red” and “V2” represent the reduced form of leupeptin). FIG. 9C shows the relative anti-AdsA antibody levels on day 49 using the two CATCH proteins indicated without any leupeptin. Mean±standard deviation shown.

FIGS. 10A-10B show the relative amounts of cleaved substrate of CATCH (4K+)-Leu (FIG. 10A) and CATCH (6D−)-Leu (FIG. 10B) measured at different concentrations (oxidized, left graphs; and reduced, right graphs). Mean±standard deviation shown.

FIGS. 11A-11B show the kinetics of the CATCH-Leu_V1 (oxidized) variants (FIG. 11A) and the CATCH-Leu_V2 (reduced) variants (FIG. 11B). Mean±standard deviation shown.

FIGS. 12A-12B show total IgG levels measured by ELISA of different treatment groups at day 21 (FIG. 12A) and day 35 (FIG. 12B). Mean±standard deviation shown.

### DETAILED DESCRIPTION

Aspects of the application relate to compositions and methods for reducing production of host antibodies against a peptide or protein (e.g., a therapeutic peptide or therapeutic protein) that is administered to the host (e.g., a mammalian host, such as a human host, which may also be referred to as a subject). As therapeutic peptides and therapeutic proteins (e.g., enzymes) have been found to be valuable in the treatment of a myriad of diseases, the production of anti-drug antibodies (ADAs), especially in the context of repeated administration or local depots remains a problem. Disclosed herein are peptides or proteins comprising a charge-complementary peptide (e.g., CATCH peptides) and a protease inhibitor, and optionally, a therapeutic protein or therapeutic peptide (e.g., supramolecular enzyme-peptide gels) for localized biotherapeutic therapeutic protein or therapeutic peptide (e.g., enzyme) delivery. Without wishing to be bound by theory, it is thought that the use of such peptides or proteins prevents antigen-processing and/or loading onto major histocompatibility complex II (MHC II), leading to a reduced humoral immune response (e.g., reduced ADA formation).

In some aspects, the application relates to a peptide or protein comprising a charge-complementary peptide (e.g., a positive peptide or a negative peptide) and a protease inhibitor. In some embodiments, one or more peptides or proteins (e.g., a positive peptide fused to a protease inhibitor, a negative peptide fused to a protease inhibitor, a therapeutic peptide, a therapeutic protein, or any combination thereof) are contacted with a target cell (e.g., a target cell in a subject, such as a mammalian subject, which includes a human subject). In some embodiments, a peptide or protein comprising a charge-complementary peptide and a protease inhibitor reduces or eliminates the activity of proteases (e.g., proteases in endolysosomal vesicles (e.g., an MHCII compartment)) in a target cell (e.g., an antigen-presenting cell) and/or prevents antigen processing, thereby reducing or eliminating the production of antibodies against, for example, a co-administered peptide or protein, such as a therapeutic peptide or therapeutic protein.

“Charge-complementary peptides,” “co-assembling peptides”, or “co-assembly peptides” refers to positive peptides and negative peptides that each comprise one or more charge-complementary segments which are capable of binding to each other under certain conditions to form larger order structures (e.g., granules, particles, hydrogels, etc.). Accordingly, co-assembling peptides comprise self-assembly properties under conditions which stimulate hydrogel formation.

Non-limiting embodiments of charge-complementary peptides are depicted in, for example, U.S. Pat. Nos. 10,906,939, 12,152,088, International Patent Application No. PCT/US2022/025426 (published as WO 2022/225,987), and International Patent Application No. PCT/US2023/065823 (published as WO 2023/201,370), which are each incorporated by reference for descriptions related to charge-complementary peptides and granules, particles, and hydrogels thereof, as well as methods of producing and administering charge-complementary peptides.

In some embodiments, the one or more charge-complementary segments comprise arrangements of charged amino acids (e.g., cationic amino acids and anionic amino acids) and hydrophobic amino acids. In some embodiments, the one or more charge-complementary segments may further comprise polar amino acids. Accordingly, as used herein “cationic amino acids” refer to residues with positively charged side (or “R” groups) groups and can include, but are not limited to, lysine, arginine, and histidine. As used herein, “anionic amino acids” refers to residues that have negatively charged side groups and can include, but are not limited to, aspartate and glutamate. As used herein “polar amino acids” refers to residue that have polar, uncharged side groups, and can include, but are not limited to, serine, threonine, cysteine, proline, asparagine, and glutamine. As used herein “hydrophobic amino acids” refers to residues that have nonpolar, aliphatic or aromatic side groups and can include, but are not limited to, glycine, alanine, valine, leucine, methionine, isoleucine, phenylalanine, tyrosine, and tryptophan. Amino acids can each be selected from natural and non-natural amino acids. Natural and non-natural amino acids can also be selected from a cationic amino acid, an anionic amino acid, a hydrophobic amino acid, or a polar amino acid. In some embodiments, the amino acids (e.g., hydrophobic, anionic, cationic, polar, etc.) can be modified, such as iodinated, fluorinated, or otherwise labeled.

In some embodiments, co-assembling peptides are at least 3 amino acids in length. In some embodiments, co-assembling peptides comprise 3-50 amino acids in length. In some embodiments, co-assembling peptides comprise 3-5, 5-8, 8-11, 11-15, 15-20, 20-25, 25-30, 30-35, 40-45, 45-50, or more amino acids in length. In some embodiments, co-assembling peptides, comprise or consist of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids in length. In some embodiments, co-assembling peptides are the same length. However, in other embodiments, the lengths of the co-assembling peptides may differ and still form interactions that drive hydrogel formation.

While, in some aspects, co-assembling peptides structures/sequences may be described herein by referring to the order of amino acids found therein relative to the N-terminus or the C-terminus of the peptide, other aspects of the present disclosure may refer to amino acids in co-assembling peptides by their position relative to other structures/sequences within the peptide. Accordingly, referring to amino acids in co-assembling peptides by describing their relative positions (e.g., as being N-terminal to, C-terminal to, flanking, etc. or as “first”, “second”, “third”, etc.) should not be considered to limit the sequence or structure of the given peptide embodiment unless explicitly stated. The following embodiments relate to co-assembling peptide structures which includes, but is not limited to, the one or more charge-complementary segments that the peptides comprise.

In some embodiments, the co-assembling functional properties of the charged, co-assembling peptides of the present disclosure stem from the structure of said peptides encoded by their primary sequences which includes a core sequence of alternating hydrophobic and hydrophilic residues. The “core sequence” refers to a charged (e.g., either positive or negative) segment of amino acids comprising either at least two hydrophobic residues which flank a cationic residue or anionic residue, or a hydrophobic residue flanked by at least one cationic residue or anionic residue and a hydrophilic residue (e.g., either polar or charged) (see Table 1). As such, both a hydrophobic core component and a charged core component, provided by the appropriate residues comprising said side chain chemistries, promote hydrogel formation (see Table 1). As such, the core structure must be sufficiently hydrophobic and charged, while other residues found in the N-terminal and/or C-terminal direction of the core structure may be cationic or anionic to further promote self-assembly.

In some embodiments, one core sequence is found in a positive peptide and/or negative peptide. In some embodiments, a positive peptide and/or a negative peptide may comprise two, three, four, or more core sequences. In some embodiments, more than one core sequence is found in a positive peptide and/or negative peptide located adjacent to each other in the primary sequence. In some embodiments, more than one core sequence is found in a positive peptide and/or negative peptide and separated by one or more (e.g., two, three, four, or more) residues. In some embodiments, a positive peptide and/or a negative peptide comprise more than one core sequence comprising the same amino acid arrangements (e.g., a positive peptide with a core sequence of KFK and KFK or, as a further example, a negative peptide with a core sequence of DFD and DFD). In some embodiments, a positive peptide and/or a negative peptide comprise more than one core sequence comprising different amino acid arrangements (e.g., a positive peptide with a core sequence of KFK and QFK or, as a further example, a negative peptide with a core sequence of DFD and QFD).

In some embodiments, a core sequence comprises one or more amino acids which are located N-terminal to the core sequence and/or C-terminal to the core sequence. In some embodiments, a core sequence comprises 1, 2, 3, 4, 5, 6, 7, 8, 9, 10, 11, 12, 13, 14, 15, or more amino acids which are located N-terminal to the core sequence and/or C-terminal to the core sequence. In some embodiments, a core sequence is flanked by sequences in the N-terminal direction and C-terminal direction (e.g., a core sequence with an equal or non-equal number of amino acids flanking it in the N-terminal and C-terminal direction). In some embodiments, the flanking sequences may be the same sequence or different sequences. In some embodiments, however, a core sequence is found at the N-terminus, the C-terminus, or both termini.

In some embodiments, a positive peptide may comprise 1-50 core sequences of at least 3 amino acids in length (see, e.g., the positively-charged core sequences as described in Table 1 herein). In some embodiments, a positive peptide may comprise 1, 2, 3, 4, 5, 6, 7, 8, 9, 10, or more (e.g., up to 15, up to 20, up to 25, etc.) core sequences of at least 3 amino acids in length, optionally wherein any or all of the core sequences are separated by at least one amino acid. In some embodiments, a positive peptide may comprise 1-3, 1-4, 1-5, 1-6, 1-7, 1-8, 1-9, 1-10, 2-7, 7-12, 12-17, 17-22, 22-27, 27-32, 32-37, 37-42, 42-47, 47-50, 3-8, 8-13, 13-18, 18-23, 23-28, 28-33, 33-38, 38-43, 43-48, 48-50, 4-9, 9-14, 14-19, 19-24, 24-29, 29-34, 34-39, 39-44, 44-49, 49-50, 5-10, or 45-50 or more core sequences of at least 3 amino acids in length, optionally wherein any or all of the core sequences are separated by at least one amino acid. In some embodiments, wherein more than one core sequence is found in a positive peptide, there may be 2, 3, 4, 5, 6, 7, 8, 9, 10, or more distinct core sequences of at least 3 amino acids in length which differ in amino acid sequence by at least one amino acid.

In some embodiments, a negative peptide may comprise 1-50 core sequences of at least 3 amino acids in length (see, e.g., the negatively-charged core sequences as described in Table 1 herein). In some embodiments, a negative peptide may comprise 1, 2, 3, 4, 5, 6, 7, 8, 9, 10, or more (e.g., up to 15, up to 20, up to 25, etc.) core sequences of at least 3 amino acids in length, optionally wherein any or all of the core sequences are separated by at least one amino acid. In some embodiments, a negative peptide may comprise 1-3, 1-4, 1-5, 1-6, 1-7, 1-8, 1-9, 1-10, 2-7, 7-12, 12-17, 17-22, 22-27, 27-32, 32-37, 37-42, 42-47, 47-50, 3-8, 8-13, 13-18, 18-23, 23-28, 28-33, 33-38, 38-43, 43-48, 48-50, 4-9, 9-14, 14-19, 19-24, 24-29, 29-34, 34-39, 39-44, 44-49, 49-50, 5-10, or 45-50 or more core sequences of at least 3 amino acids in length, optionally wherein any or all of the core sequences are separated by at least one amino acid. In some embodiments, wherein more than one core sequence is found in a negative peptide, there may be 2, 3, 4, 5, 6, 7, 8, 9, 10, or more distinct core sequences of at least 3 amino acids in length which differ in amino acid sequence by at least one amino acid.

Apart from the co-assembling peptide species provided herein, other species within the genus of co-assembling peptides may be designed using the teachings provided herein. For example, charge-complementarity between the positive and negative peptides is necessary for hydrogel formation. Accordingly, a minimum of two charged residues (e.g., at least one cationic residue in the positive peptide and at least one anionic residue in the negative peptide) may be necessary to electrostatically repress similarly charged peptides and prevent their association.

Additionally, the present disclosure describes types of amino acid substitutions that can be made to engineer co-assembling peptides. For example, for the hydrophobic component of the core sequence, phenylalanine could be switched to tryptophan but could not be switched to a polar or charged residue (see Table 1). In a separate example, the charged residues of the core component could comprise arginine instead of lysine and/or aspartate instead of glutamate (see Table 1). For other residues found either in the core structure or other positions of the charged peptides, polar residues could be swapped as appropriate (e.g., asparagine substituted for glutamine; see Table 1).

Table 1 shown below provides primary amino acid sequences that are found in co-assembling peptides. For example, Table 1 describes specific examples of co-assembling peptide sequences and core sequences that may be used to design other co-assembling peptides capable of forming hydrogels.

TABLE 1
Non-Limiting Embodiments of Co-assembling
Peptide Core Sequences

| | Primary Sequence |
|---|---|
| Species | (N-term. to C-term.) |
| Examples of | KFK, KWK, KYK, KAK, KGK, KMK, KVK, |
| positively- | KLK, KIK, KFR, KWR, KYR, KAR, KGR, |
| charged core | KMR, KVR, KLR, KIR, KFH, KWH, KYH, |
| sequences (also | KAH, KGH, KMH, KVH, KLH, KIH, KFS, |
| referred to as | KWS, KYS, KAS, KGS, KMS, KVS, KLS, |
| cationic core | KIS, KFT, KWT, KYT, KAT, KGT, KMT, |
| sequences) | KVT, KLT, KIT, KFC, KWC, KYC, KAC, |
| | KGC, KMC, KVC, KLC, KIC, KFN, KWN, |
| | KYN, KAN, KGN, KMN, KVN, KLN, KIN, |
| | KFQ, KWQ, KYQ, KAQ, KGQ, KMQ, KVQ, |
| | KLQ, KIQ, RFK, RWK, RYK, RAK, RGK, |
| | RMK, RVK, RLK, RIK, RFR, RWR, RYR, |
| | RAR, RGR, RMR, RVR, RLR, RIR, RFH, |
| | RWH, RYH, RAH, RGH, RMH, RVH, RLH, |
| | RIH, RFS, RWS, RYS, RAS, RGS, RMS, |
| | RVS, RLS, RIS, RFT, RWI, RYT, RAT, |
| | RGT, RMT, RVT, RLT, RIT, RFC, RWC, |
| | RYC, RAC, RGC, RMC, RVC, RLC, RIC, |
| | RFN, RWN, RYN, RAN, RGN, RMN, RVN, |
| | RLN, RIN, RFQ, RWQ, RYQ, RAQ, RGQ, |
| | RMQ, RVQ, RLQ, RIQ, HFK, HWK, HYK, |
| | HAK, HGK, HMK, HVK, HLK, HIK, HFR, |
| | HWR, HYR, HAR, HGR, HMR, HVR, HLR, |
| | HIR, HFH, HWH, HYH, HAH, HGH, HMH, |
| | HVH, HLH, HIH, HFS, HWS, HYS, HAS, |
| | HGS, HMS, HVS, HLS, HIS, HFT, HWT, |
| | HYT, HAT, HGT, HMT, HVT, HLT, HIT, |
| | HFC, HWC, HYC, HAC, HGC, HMC, HVC, |
| | HLC, HIC, HFN, HWN, HYN, HAN, HGN, |
| | HMN, HVN, HLN, HIN, HFQ, HWQ, HYQ, |
| | HAQ, HGQ, HMQ, HVQ, HLQ, HIQ, SFK, |
| | SWK, SYK, SAK, SGK, SMK, SVK, SLK, |
| | SIK, SFH, SWH, SYH, SAH, SGH, SMH, |
| | SVH, SLH, SIH, SFR, SWR, SYR, SAR, |
| | SGR, SMR, SVR, SLR, SIR, TFK, TWK, |
| | TYK, TAK, TGK, TMK, TVK, TLK, TIK, |
| | TFH, TWH, TYH, TAH, TGH, TMH, TVH, |
| | TLH, TIH, TFR, TWR, TYR, TAR, TGR, |
| | TMR, TVR, TLR, TIR, CFK, CWK, CYK, |
| | CAK, CGK, CMK, CVK, CLK, CIK, CFH, |
| | CWH, CYH, CAH, CGH, CMH, CVH, CLH, |
| | CIH, CFR, CWR, CYR, CAR, CGR, CMR, |
| | CVR, CLR, CIR, QFK, QWK, QYK, QAK, |
| | QGK, QMK, QVK, QLK, QIK, QFH, QWH, |
| | QYH, QAH, QGH, QMH, QVH, QLH, QIH, |
| | QFR, QWR, QYR, QAR, QGR, QMR, QVR, |
| | QLR, QIR, NFK, NWK, NYK, NAK, NGK, |
| | NMK, NVK, NLK, NIK, NFH, NWH, NYH, |
| | NAH, NGH, NMH, NVH, NLH, NIH, NFR, |
| | NWR, NYR, NAR, NGR, NMR, NVR, NLR, |
| | NIR, FKF, FRF, FHF, FKW, FRW, FHW, |
| | FKY, FRY, FHY, FKA, FRA, FHA, FKI, |
| | FRI, FHI, FKL, FRL, FHL, FKV, FRV, |
| | FHV, FKM, FRM, FHM, FKG, FRG, FHG, |
| | WKF, WRF, WHF, WKW, WRW, WHW, WKY, |
| | WRY, WHY, WKA, WRA, WHA, WKI, WRI, |
| | WHI, WKL, WRL, WHL, WKV, WRV, WHV, |
| | WKM, WRM, WHM, WKG, WRG, WHG, YKF, |
| | YRF, YHF, YKW, YRW, YHW, YKY, YRY, |
| | YHY, YKA, YRA, YHA, YKI, YRI, YHI, |
| | YKL, YRL, YHL, YKV, YRV, YHV, YKM, |
| | YRM, YHM, YKG, YRG, YHG, AKF, ARF, |
| | AHF, AKW, ARW, AHW, AKY, ARY, AHY, |
| | AKA, ARA, AHA, AKJ, ARI, AHI, AKL, |
| | ARL, AHL, AKV, ARV, AHV, AKM, ARM, |
| | AHM, AKG, ARG, AHG, IKF, IRF, IHF, |
| | IQF, INF, IKW, IRW, IHW, IKY, IRY, |
| | IHY, IKA, IRA, IHA, IKI, IRI, IHI, |
| | IKL, IRL, IHL, IKV, IRV, IHV, IKM, |
| | IRM, IHM, IKG, IRG, IHG, LKF, LRF, |
| | LHF, LKW, LRW, LHW, LKY, LRY, LHY, |
| | LKA, LRA, LHA, LKI, LRI, LHI, LKL, |
| | LRL, LHL, LKV, LRV, LHV, LKM, LRM, |
| | LHM, LKG, LRG, LHG, VKF, VRF, VHI, |
| | VKW, VRW, VHW, VKY, VRY, VHY, VKA, |
| | VRA, VHA, VKI, VRI, VHI, VKL, VRL, |
| | VHL, VKV, VRV, VHV, VKM, VRM, VHM, |
| | VKG, VRG, VHG, MKF, MRF, MHF, MKW, |
| | MRW, MHW, MKY, MRY, MHY, MKA, MRA, |
| | MHA, MKI, MRI, MHI, MKL, MRL, MHL, |
| | MKV, MRV, MHV, MKM, MRM, MHN, MKG, |
| | MRG, MHG, GKF, GRF, GHF, GKW, GRW, |
| | GHW, GKY, GRY, GHY, GKA, GRA, GHA, |
| | GKI, GRI, GHI, GKL, GRL, GHL, GKV, |
| | GRV, GHV, GKM, GRM, GHM, GKG, GRG, |
| | GHG |
| Examples of | EFE, EWE, EYE, EAE, EGE, EME, EVE, |
| negatively- | ELE, EIE, EFS, EWS, EYS, EAS, EGS, |
| charged core | EMS, EVS, ELS, EIS, EFT, EWT, EYT, |
| sequences (also | EAT, EGT, EMT, EVT, ELT, EIT, EFC, |
| referred to as | EWC, EYC, EAC, EGC, EMC, EVC, ELC, |
| anionic core | EIC, EFN, EWN, EYN, EAN, EGN, EMN, |
| sequences) | EVN, ELN, EIN, EFQ, EWQ, EYQ, EAQ, |
| | EGQ, EMQ, EVQ, ELQ, EIQ, DFD, DWD, |
| | DYD, DAD, DGD, DMD, DVD, DLD, DID, |
| | DFS, DWS, DYS, DAS, DGS, DMS, DVS, |
| | DLS, DIS, DFT, DWT, DYT, DAT, DGT, |
| | DMT, DVT, DLT, DIT, DFC, DWC, DYC, |
| | DAC, DGC, DMC, DVC, DLC, DIC, DFN, |
| | DWN, DYN, DAN, DGN, DMN, DVN, DIN, |
| | DIN, DFQ, DWQ, DYQ, DAQ, DGQ, DMQ, |
| | DVQ, DLQ, DIQ, SFE, SWE, SYE, SAE, |
| | SGE, SME, SVE, SLE, SIE, SED, SWD, |
| | SYD, SAD, SGD, SMD, SVD, SLD, SID, |
| | TFE, TWE, TYE, TAE, TGE, TME, TVE, |
| | TLE, TIE, TFD, TWD, TYD, TAD, TGD, |
| | TMD, TVD, TLD, TID, CFE, CWE, CYE, |
| | CAE, CGE, CME, CVE, CLE, CIE, CFD, |
| | CWD, CYD, CAD, CGD, CMD, CVD, CLD, |
| | CID, QFE, QWE, QYE, QAE, QGE, QME, |
| | QVE, QLE, QIE, QFD, QWD, QYD, QAD, |
| | QGD, QMD, QVD, QLD, QID, NFE, NWE, |
| | NYE, NAE, NGE, NME, NVE, NLE, NIE, |
| | NFD, NWD, NYD, NAD, NGD, NMD, NVD, |
| | NLD, NID, FEF, FDF, FEW, FDW, FEY, |
| | FDY, FEA, FDA, FEI, FDI, FEL, FDL, |
| | FEV, FDV, FEM, FDM, FEG, FDG, WEF, |
| | WDF, WEW, WDW, WEY, WDY, WEA, WDA, |
| | WEI, WDI, WEL, WDL, WEV, WDV, WEM, |
| | WDM, WEG, WDG, YEF, YDF, YEW, YDW, |
| | YEY, YDY, YEA, YDA, YEI, YDI, YEL, |
| | YDL, YEV, YDV, YEM, YDM, YEG, YDG, |
| | AEF, ADF, AEW, ADW, AEY, ADY, AEA, |
| | ADA, AEI, ADI, AEL, ADL, AEV, ADV, |
| | AEM, ADM, AEG, ADG, IEF, IDF, IEW, |
| | IDW, IEY, IDY, IEA, IDA, IEI, IDI, |
| | IEL, IDL, IEV, IDV, IEM, IDM, IEG, |
| | IDG, LEF, LDF, LEW, LDW, LEY, LDY, |
| | LEA, LDA, LEI, LDI, LEL, LDL, LEV, |
| | LDV, LEM, LDM, LEG, LDG, VEF, VDF, |
| | VEW, VDW, VEY, VDY, VEA, VDA, VEI, |
| | VDI, VEL, VDL, VEV, VDV, VEM, VDM, |
| | VEG, VDG, MEF, MDF, MEW, MDW, MEY, |
| | MDY, MEA, MDA, MEI, MDI, MEL, MDL, |
| | MEV, MDV, MEM, MDM, MEG, MDG, GEF, |
| | GDF, GEW, GDW, GEY, GDY, GEA, GDA, |
| | GEI, GDI, GEL, GDL, GEV, GDV, GEM, |
| | GDM, GEG, GDG |

In some embodiments, a positive peptide can include at least 3 amino acids, at least 4 amino acids, at least 5 amino acids, at least 6 amino acids, or at least 7 amino acids which can each be independently selected from a cationic amino acid and a hydrophobic amino acid, and wherein at least one amino acid of said amino acids can be a cationic amino acid and at least one said amino acids can be a hydrophobic amino acid. In some embodiments, the positive peptide may comprise the at least 3 amino acids, the at least 4 amino acids, the at least 5 amino acids, or the at least 7 amino acids which are arranged in alternating order by side chain chemistry (e.g., cationic amino acid-hydrophobic amino acid-cationic amino acid, such as to be part of a cationic core sequence that binds to an anionic core sequence).

In some embodiments, a positive peptide comprises a cationic core sequence comprising at least 3 amino acids, wherein a first hydrophobic amino acid is flanked by at least one cationic amino acid and a first hydrophilic amino acid which is either cationic or polar. In some embodiments, the positive peptide further comprises a second hydrophobic amino acid N-terminal to the cationic core sequence, a third hydrophobic amino acid C-terminal to the cationic core sequence, or the second hydrophobic amino acid and the third hydrophobic amino acid. In some embodiments, the positive peptide further comprises a second hydrophilic amino acid N-terminal to the second hydrophobic amino acid and a third hydrophilic amino acid C-terminal to the third hydrophobic amino acid, wherein the second hydrophilic amino acid and the third hydrophilic amino acid are either cationic or polar. In some embodiments, the positive peptide further comprises one or more (e.g., 1, 2, 3, 4, or more) cationic or polar amino acids that are found N-terminal to and/or C-terminal to the second hydrophilic amino acid and the third hydrophilic amino acid.

In some embodiments, a positive peptide comprises a cationic core sequence comprising at least 3 amino acids, wherein a first hydrophobic amino acid and a second hydrophobic amino acid flank a cationic amino acid. In some embodiments, the positive peptide further comprises a first hydrophilic amino acid N-terminal to the cationic core sequence, a second hydrophilic amino acid C-terminal to the cationic core sequence, or both the first hydrophilic amino acid and the second hydrophilic amino acid, wherein the first hydrophilic amino and the second hydrophilic amino acid are either cationic or polar. In some embodiments, the positive peptide further comprises a third hydrophobic amino acid N-terminal to the first hydrophilic amino and a fourth hydrophobic amino acid C-terminal to the second hydrophilic amino acid. In some embodiments, the positive peptide further comprises one or more (e.g., 1, 2, 3, 4, or more) hydrophilic amino acids that are found N-terminal to third hydrophobic amino acid and/or C-terminal to the fourth hydrophobic amino acid.

A positive peptide can include at least 3 amino acids wherein each amino acid can be independently selected from a positively charged amino acid and a hydrophobic amino acid, and wherein at least one amino acid is a positively charged amino acid and at least one amino acid is a hydrophobic amino acid. A positive peptide can include at least 4 amino acids wherein each amino acid may be independently selected from a positively charged amino acid and a hydrophobic amino acid, and wherein at least one amino acid can be a positively charged amino acid and at least one amino acid can be a hydrophobic amino acid. A positive peptide can include at least 5 amino acids wherein each be independently selected from a positively charged amino acid and a hydrophobic amino acid, and wherein at least one amino acid can be a positively charged amino acid and at least one amino acid can be a hydrophobic amino acid. A positive peptide can include at least 7 amino acids wherein each be independently selected from a positively charged amino acid and a hydrophobic amino acid, and wherein at least one amino acid can be a positively charged amino acid and at least one amino acid can be a hydrophobic amino acid. The positively charged amino acids (cationic amino acids) can each be independently selected from the group of lysine, histidine, and arginine. The hydrophobic amino acids can each be independently selected from the group consisting of: glycine, alanine, valine, leucine, methionine, isoleucine, phenylalanine, tyrosine, and tryptophan. In some embodiments, the peptide having a net positive charge can have at least 11 amino acids, wherein each can be a polar or a cationic amino acid.

In some embodiments, the positive peptide segment comprises a sequence of QQKFKFKFKQQ (SEQ ID NO: 1) (also referred to herein as CATCH (4K+)), KQKFKFKFKQK (SEQ ID NO: 3) (also referred to herein as CATCH (6K+)), KQQFKFKFKQQ (SEQ ID NO: 5), QQKFQFQFKQQ (SEQ ID NO: 6) (also referred to herein as CATCH (2+)), or KQQFKFKFQQK (SEQ ID NO: 8) (also referred to herein as CATCH (*4+)).

In some embodiments, a negative peptide can include at least 3 amino acids, at least 4 amino acids, at least 5 amino acids, at least 6 amino acids, or at least 7 amino acids which can each be independently selected from an anionic amino acid and a hydrophobic amino acid, and wherein at least one amino acid of said amino acids can be an anionic amino acid and at least one said amino acids can be a hydrophobic amino acid. In some embodiments, the negative peptide may comprise the at least 3 amino acids, the at least 4 amino acids, the at least 5 amino acids, or the at least 7 amino acids which are arranged in alternating order based on side chain chemistry (e.g., anionic amino acid-hydrophobic amino acid-anionic amino acid, such as to be part of an anionic core sequence).

In some embodiments, a negative peptide comprises an anionic core sequence comprising at least 3 amino acids, wherein a first hydrophobic amino acid is flanked by at least one anionic amino acid and a first hydrophilic amino acid which is either anionic or polar. In some embodiments, the negative peptide further comprises a second hydrophobic amino acid N-terminal to the anionic core sequence, a third hydrophobic amino acid C-terminal to the anionic core sequence, or the second hydrophobic amino acid and the third hydrophobic amino acid. In some embodiments, the negative peptide further comprises a second hydrophilic amino acid N-terminal to the second hydrophobic amino acid and a third hydrophilic amino acid C-terminal to the third hydrophobic amino acid, wherein the second hydrophilic amino acid and the third hydrophilic amino acid are either anionic or polar. In some embodiments, the negative peptide further comprises one or more (e.g., 1, 2, 3, 4, or more) anionic or polar amino acids that are found N-terminal to and/or C-terminal to the second hydrophilic amino acid and the third hydrophilic amino acid.

In some embodiments, a negative peptide comprises an anionic core sequence comprising at least 3 amino acids, wherein a first hydrophobic amino acid and a second hydrophobic amino acid flank an anionic amino acid. In some embodiments, the negative peptide further comprises a first hydrophilic amino acid N-terminal to the anionic core sequence, a second hydrophilic amino acid C-terminal to the anionic core sequence, or both the first hydrophilic amino acid and the second hydrophilic amino acid, wherein the first hydrophilic amino and the second hydrophilic amino acid are either anionic or polar. In some embodiments, the negative peptide further comprises a third hydrophobic amino acid N-terminal to the first hydrophilic amino and a fourth hydrophobic amino acid C-terminal to the second hydrophilic amino acid. In some embodiments, the negative peptide further comprises one or more (e.g., 1, 2, 3, 4, or more) hydrophilic amino acids that are found N-terminal to third hydrophobic amino acid and/or C-terminal to the fourth hydrophobic amino acid.

In some embodiments, a charge-complementary peptide is a positively charged peptide. In some embodiments, a positively charged peptide comprises an amino acid sequence of SEQ ID NO: 1, 3, 5, 6, or 8. In some embodiments, a charge-complementary peptide is a negatively charged peptide. In some embodiments, a negatively charged peptide comprises an amino acid sequence of EQEFEFEFEQE (SEQ ID NO: 2) (also referred to herein as CATCH (6E−)), DQDFDFDFDQD (SEQ ID NO: 4) ((also referred to herein as CATCH (6D−)), QQEFEFEFEQQ (SEQ ID NO: 7) (also referred to herein as CATCH (4−)), QQEFQFQFEQQ (SEQ ID NO: 9) (also referred to herein as CATCH (2−)). In some embodiments, a composition comprises charge-complementary peptides, including at least one positively charged peptide and at least one negatively charged peptide, thereby forming a supramolecular structure (e.g., a particle, a granule, or a hydrogel), wherein one or more of the charge-complementary peptides are fused to a protease inhibitor and, optionally wherein one or more of the charge-complementary peptides are fused (e.g., linked) to a therapeutic protein or therapeutic peptide.

In some embodiments, the positively charged peptide is SEQ ID NO: 1 and the negatively charged peptide is SEQ ID NO: 2. In some embodiments, the positively charged peptide is SEQ ID NO: 1 and the negatively charged peptide is SEQ ID NO: 4. In some embodiments, the positively charged peptide is SEQ ID NO: 1 and the negatively charged peptide is SEQ ID NO: 7. In some embodiments, the positively charged peptide is SEQ ID NO: 1 and the negatively charged peptide is SEQ ID NO: 9. In some embodiments, the positively charged peptide is SEQ ID NO: 3 and the negatively charged peptide is SEQ ID NO: 2. In some embodiments, the positively charged peptide is SEQ ID NO: 3 and the negatively charged peptide is SEQ ID NO: 4. In some embodiments, the positively charged peptide is SEQ ID NO: 3 and the negatively charged peptide is SEQ ID NO: 7. In some embodiments, the positively charged peptide is SEQ ID NO: 3 and the negatively charged peptide is SEQ ID NO: 9. In some embodiments, the positively charged peptide is SEQ ID NO: 5 and the negatively charged peptide is SEQ ID NO: 2. In some embodiments, the positively charged peptide is SEQ ID NO: 5 and the negatively charged peptide is SEQ ID NO: 4. In some embodiments, the positively charged peptide is SEQ ID NO: 5 and the negatively charged peptide is SEQ ID NO: 7. In some embodiments, the positively charged peptide is SEQ ID NO: 5 and the negatively charged peptide is SEQ ID NO: 9. In some embodiments, the positively charged peptide is SEQ ID NO: 6 and the negatively charged peptide is SEQ ID NO: 2. In some embodiments, the positively charged peptide is SEQ ID NO: 6 and the negatively charged peptide is SEQ ID NO: 4. In some embodiments, the positively charged peptide is SEQ ID NO: 6 and the negatively charged peptide is SEQ ID NO: 7. In some embodiments, the positively charged peptide is SEQ ID NO: 6 and the negatively charged peptide is SEQ ID NO: 9. In some embodiments, the positively charged peptide is SEQ ID NO: 8 and the negatively charged peptide is SEQ ID NO: 2. In some embodiments, the positively charged peptide is SEQ ID NO: 8 and the negatively charged peptide is SEQ ID NO: 4. In some embodiments, the positively charged peptide is SEQ ID NO: 8 and the negatively charged peptide is SEQ ID NO: 7. In some embodiments, the positively charged peptide is SEQ ID NO: 8 and the negatively charged peptide is SEQ ID NO: 9.

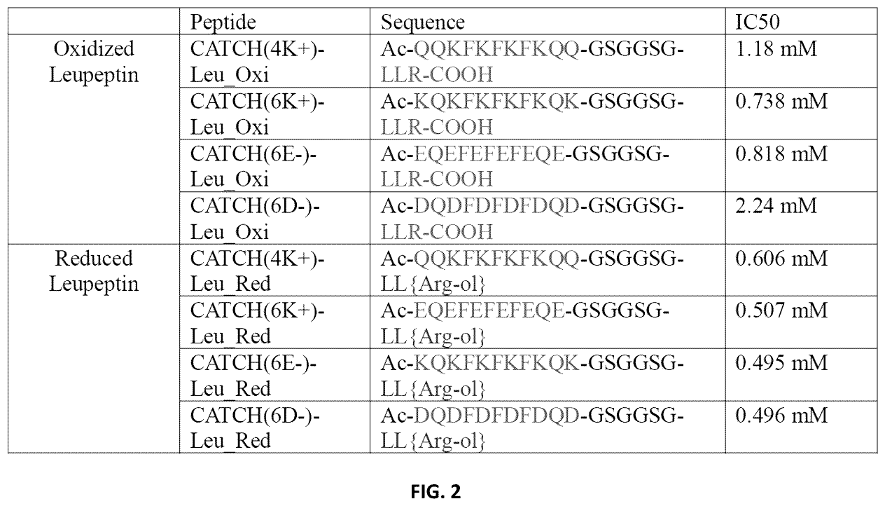

In some embodiments, a charge-complementary peptide comprises a positive peptide (e.g., a positive CATCH peptide, such as a positive peptide comprising an amino acid sequence shown in FIG. 2). In some embodiments, a charge-complementary peptide comprises a negative peptide (e.g., a negative CATCH peptide, such as a negative peptide comprising an amino acid sequence shown in FIG. 2).

In some embodiments, a peptide or protein comprises one or more (e.g., one, two, three, or more than three) protease inhibitors (e.g., wherein the one or more protease inhibitors are fused to a charged-complementary peptide or to a therapeutic protein or therapeutic peptide). As used herein, “protease inhibitor” refers to a molecule that at least partially inhibits the function of one or more protein-based enzymes that cleave other proteins. Examples of protease inhibitors include, but are not limited to, aprotinin, bestatin, chymostatin, leupeptin, alpha-2-macroglobulin, pepstatin, phenylmethanesulfonyl fluoride, N-ethylmaleimide, ethylenediaminetetraacetid acid, antithrombin, or combinations thereof. In some embodiments, the protease inhibitor is in its oxidized form. In some embodiments, the protease inhibitor is in its reduced form. In some embodiments, a protease inhibitor is an inhibitor of a serine protease, a threonine protease, and/or a cysteine protease. In some embodiments, a protease inhibitor is an inhibitor of an aspartic protease. In some embodiments, a protease inhibitor is an inhibitor of a lysosomal protease. In some embodiments, a protease inhibitor inhibits cathepsins. In some embodiments, a protease inhibitor comprises a leupeptin or a modified form thereof. Leupeptin, Ac-Leu-Leu-Arg-CHO, is a naturally occurring tripeptide which inhibits a variety of proteases, and may be able to prevent antigen processing. In some embodiments, a protease inhibitor comprises an oxidized leupeptin (see, e.g., the oxidized leupeptins described in FIG. 2). In some embodiments, a protease inhibitor comprises a reduced leupeptin (see, e.g., the reduced leupeptins described in FIG. 2). In some embodiments, a protease inhibitor comprises Pepstatin A or a modified form thereof (e.g., wherein the protease inhibitor comprises the amino acid sequence of: {Iso-Val} {Val} {Val} {Sta} {Ala} {Sta} (SEQ ID NO: 18), wherein “Iso-Val” refers to isovaline and “Sta” refers to statine).

In some embodiments, a protease inhibitor is fused directly to a charge-complementary peptide (e.g., via a covalent bond at the N-terminus and/or a covalent bond at the C-terminus of the charge complementary peptide) or fused directly to a therapeutic protein or therapeutic peptide (e.g., via a covalent bond at the N-terminus and/or a covalent bond at the C-terminus of the therapeutic protein or therapeutic peptide). In some embodiments, a protease inhibitor is fused to a charge-complementary peptide via a linker (e.g., wherein the linker is connected to the N-terminus or the C-terminus of the charge-complementary peptide). In some embodiments, a protease inhibitor is fused to a therapeutic protein or therapeutic peptide via a linker (e.g., wherein the linker is connected to the N-terminus or the C-terminus of the therapeutic protein or therapeutic peptide). In some embodiments, a linker is an amino acid linker, such as a flexible linker (e.g., a hydrophilic linker, such as a glycine-serine-rich linker, which includes, but is not limited to, a linker glycine-serine linker described in FIG. 2).

As used herein, “linker” can refer to any amino acid or peptide that can be optionally included between a positive or negative peptide segment and a protease inhibitor, a therapeutic protein, a therapeutic peptide, or any combination thereof. The linker can range in length from about 1 to about 60 amino acids in length (e.g., 1-5, 5-10, 10-20, 10-25, 10-30, 15-20, 15-25, 15-30, 20-30, 3-40, or 10-50 amino acids in length). In some embodiments, linker is 1, 3, 4, 5, 6, 7, 8, 9, or 10 amino acids in length. The linker can be composed of any of the 20 naturally occurring amino acids and be present in any arrangement that does not otherwise perturb the peptide segment assembly or cargo protein activity.

In some embodiments, a protease inhibitor is fused to the N-terminus and/or the C-terminus of a charge-complementary peptide. In some embodiments, a protease inhibitor is fused to the N-terminus and/or the C-terminus of a positively charged peptide. In some embodiments, a protease inhibitor is fused to the N-terminus and/or the C-terminus of a negatively charged peptide. In some embodiments, a leupeptin or a modified form thereof is fused to the N-terminus and/or the C-terminus of a charge-complementary peptide. In some embodiments, a leupeptin or a modified form thereof is fused to the N-terminus and/or the C-terminus of a positively charged peptide. In some embodiments, a leupeptin or a modified form thereof is fused to the N-terminus and/or the C-terminus of a negatively charged peptide.

In some embodiments, a protease inhibitor is fused to the N-terminus and/or the C-terminus of a therapeutic protein or therapeutic peptide. In some embodiments, a leupeptin or a modified form thereof is fused to the N-terminus and/or the C-terminus of a therapeutic protein or therapeutic peptide.

In some embodiments, the peptide or protein comprising a charge-complementary peptide and a protease inhibitor further comprises a therapeutic protein or therapeutic peptide. A therapeutic protein or therapeutic peptide is a protein or peptide which leads to a physiological change (e.g., in a cell, such as a cell in a subject) that is associated with or expected to at least partially, if not fully, remedy at least one symptom associated with a disease, disorder, or condition. In some embodiments, the therapeutic protein or therapeutic peptide comprises a cytokine, a chemokine, a cell-targeting peptide or protein, an organelle-targeting peptide or protein, a tissue-targeting peptide or protein, a cell-penetrating peptide or protein, an antibody (e.g., a monoclonal antibody, a polyclonal antibody, a nanobody, a single-chain antibody, such as an scFv, etc.), an antigen-binding fragment, an enzyme or an enzymatic domain (e.g., a protease, such as a protease that is not targeted by a co-administered protease inhibitor), signaling protein, polymerase, transcriptional regulator, nuclease, RNA-guided nuclease, metabolic enzyme, kinase, phosphatase, glycosylase, DNA ligase, ubiquitin ligase, transferase, lipid transferase, methyltransferase, acetyltransferase, SUMO transferase, protease, foldase, reductase, lyase, dehydrogenase, carboxylase, decarboxylase, phosphorylase, dephosphorylase, synthase, such as Adenosine synthase A, and hydrolase, etc.), a proteinaceous enzyme substrate, a glycoprotein, a lipoprotein, a secreted protein, an extracellular matrix protein or a fragment thereof, a transmembrane receptor or a fragment thereof, a toxin or a fragment thereof, hormones, receptors, a peptibody, a growth factor, a clotting factor, an activating or inhibitory peptide (e.g., a peptide capable of targeting a cell surface receptor or ion channel), a thrombolytic, a bone morphogenetic protein, an Fc-fusion protein, an anticoagulant, a nucleic acid-binding protein (e.g., transcription regulators and/or translation regulators), a detectable marker, or a reporter. In some embodiments, the therapeutic protein or therapeutic peptide is an enzyme. In some embodiments, the enzyme is selected from: indoleamine 2,3-dioxygenase, uricase, adenosine synthase A, luciferase, or a combination thereof. In some embodiments, the enzyme is adenosine synthase A. In some embodiments, the therapeutic peptide or therapeutic protein is not an antigen. In some embodiments, the therapeutic peptide or therapeutic protein is not a viral antigen. In some embodiments, the therapeutic peptide or therapeutic protein is not a bacterial antigen. In some embodiments, the therapeutic protein or therapeutic peptide is not a microbial antigen. In some embodiments, the therapeutic protein or therapeutic peptide is not a fungal peptide. In some embodiments, the therapeutic peptide or therapeutic protein is not an antigen, wherein the antigen is the active ingredient of a vaccine (e.g., a prophylactic vaccine). In some embodiments, the therapeutic peptide or therapeutic protein is not a vaccine antigen (e.g., an antigen or immunogenic fragment thereof that is included in vaccine, such as a prophylactic vaccine).

In some embodiments, the therapeutic protein or therapeutic peptide is fused to the N-terminus and/or the C-terminus of a charge-complementary peptide. In some embodiments, the therapeutic protein or therapeutic peptide is fused to the N-terminus and/or the C-terminus of a positively charged peptide (e.g., the positively charged peptide or a plurality of positive peptides each further comprise a therapeutic protein or therapeutic peptide). In some embodiments, the therapeutic protein or therapeutic peptide is fused to the N-terminus and/or the C-terminus of a negatively charged peptide (e.g., the negatively charged peptide or a plurality of negative peptides each further comprise a therapeutic protein or therapeutic peptide). In some embodiments, the plurality of positive peptides and the plurality of negative peptides each further comprise a therapeutic protein or therapeutic peptide (e.g., a therapeutic protein or therapeutic peptide is fused to the N-terminus and/or the C-terminus of each negative peptide and each positive peptide). In some embodiments, the therapeutic protein or therapeutic peptide is fused to a charge-complementary peptide directly (e.g., wherein the therapeutic protein or therapeutic peptide is linked by a covalent bond to the N-terminus or the C-terminus of the charge-complementary peptide). In some embodiments, the therapeutic protein or therapeutic peptide is fused to a charge-complementary peptide via a linker (e.g., wherein the therapeutic protein or therapeutic peptide is connected to a linker and the linker is connected to the N-terminus or the C-terminus of the charge-complementary peptide). In some embodiments, a linker is an amino acid linker, such as a flexible linker (e.g., a hydrophilic linker, such as a glycine-serine-rich linker).

In some embodiments, the therapeutic protein or therapeutic peptide is fused to the N-terminus and/or the C-terminus of a protease inhibitor. In some embodiments, the therapeutic protein or therapeutic peptide is fused to a protease inhibitor directly (e.g., wherein the therapeutic protein or therapeutic peptide is linked by a covalent bond to the N-terminus or the C-terminus of the protease inhibitor). In some embodiments, the therapeutic protein or therapeutic peptide is fused to a protease inhibitor via a linker (e.g., wherein the therapeutic protein or therapeutic peptide is connected to a linker and the linker is connected to the N-terminus or the C-terminus of the protease inhibitor). In some embodiments, a linker is an amino acid linker, such as a flexible linker (e.g., a hydrophilic linker, such as a glycine-serine-rich linker).

The therapeutic protein or therapeutic peptide can be coupled to the positive or negative peptide or therapeutic protein or therapeutic peptide using standard molecular biology and recombinant DNA technology techniques. For example, a fusion peptide segment containing the therapeutic protein or therapeutic peptide, can be produced from a recombinant DNA construct containing DNA encoding the negative or positive peptide (and/or protease inhibitor) operatively coupled with DNA encoding the therapeutic protein or therapeutic peptide and any optional linker. The DNA encoding the negative or positive peptide (and/or protease inhibitor) can be operatively coupled to the therapeutic protein or therapeutic peptide and any optional linker such that the therapeutic protein or therapeutic peptide is translated in-frame with the negative or positive peptide (and/or protease inhibitor).

The therapeutic protein or therapeutic peptide, in some embodiments, comprises a pharmaceutically relevant protein (a protein that can be effective to prevent or treat a disease or symptom thereof in a subject), a cell- or tissue-targeting protein, an antibody or fragment thereof, an enzyme, a growth factor, a cytokine, a chemokine, an extracellular matrix protein or fragment thereof, a transmembrane receptor or fragment thereof, a toxin or a fragment thereof, and a transcription factor or fragment thereof.

In some embodiments, a composition (e.g., a pharmaceutical composition) comprises a protein comprising a charge-complementary peptide and protease inhibitor (e.g., wherein the charge-complementary peptide comprises a positively charged peptide or a negatively charged peptide). In some embodiments, a composition (e.g., a pharmaceutical composition) comprises a plurality of proteins (e.g., two, three, four, five, or more than five proteins), wherein each protein of the plurality comprises a charge-complementary peptide (e.g., a positively charged peptide or a negatively charged peptide) and a protease inhibitor. In some embodiments, a composition (e.g., a pharmaceutical composition) comprises a positively charged peptide and a negatively charged peptide that are capable of co-assembling (e.g., under one or more conditions described herein) into an assembly (e.g., a supramolecular structure, such as a granule, particle, or hydrogel), wherein the positively charged peptide and/or the negatively charged peptide is fused to a protease inhibitor (e.g., fused at the N- and/or C-terminus of the charge-complementary peptide), such as directly fused or indirectly fused (e.g., fused via a linker). In some embodiments, at least 10% of the total amount of a charge-complementary peptide in a composition (e.g., at least 10% of the positively charged peptide or at least 10% of the negatively charged peptide in a composition) is fused to a protease inhibitor. In some embodiments, 10-100% of the total amount of a charge-complementary peptide in a composition (e.g., 10%-25%, 25%-50%, 50%-75%, or 75%-100% of the total amount of a positively charged peptide or a negatively charged peptide in a composition) is fused to a protease inhibitor. In some embodiments, 100% of the total amount of a charge-complementary peptide in a composition is fused to a protease inhibitor.

In some embodiments, a composition (e.g., a pharmaceutical composition) comprises a protein comprising a charge-complementary peptide, a therapeutic protein or therapeutic peptide, and protease inhibitor (e.g., wherein the charge-complementary peptide comprises a positively charged peptide or a negatively charged peptide). In some embodiments, a composition (e.g., a pharmaceutical composition) comprises a plurality of proteins (e.g., two, three, four, five, or more than five proteins), wherein each protein of the plurality comprises a charge-complementary peptide (e.g., a positively charged peptide or a negatively charged peptide), a therapeutic protein or therapeutic peptide, and a protease inhibitor. In some embodiments, a composition (e.g., a pharmaceutical composition) comprises a positively charged peptide and a negatively charged peptide that are capable of co-assembling (e.g., under one or more conditions described herein) into an assembly (e.g., a supramolecular structure, such as a granule, particle, or hydrogel), wherein the positively charged peptide and/or the negatively charged peptide is fused to a protease inhibitor and/or a therapeutic protein or therapeutic peptide (e.g., fused at the N- and/or C-terminus of the charge-complementary peptide), such as directly fused or indirectly fused (e.g., fused via a linker). In some embodiments, at least 10% of the total amount of a charge-complementary peptide in a composition (e.g., at least 10% of the positively charged peptide or at least 10% of the negatively charged peptide in a composition) is fused to a protease inhibitor and/or a therapeutic protein or therapeutic peptide. In some embodiments, 10-100% of the total amount of a charge-complementary peptide in a composition (e.g., 10%-25%, 25%-50%, 50%-75%, or 75%-100% of the total amount of a positively charged peptide or a negatively charged peptide in a composition) is fused to a protease inhibitor and/or a therapeutic protein or therapeutic peptide. In some embodiments, 100% of the total amount of a charge-complementary peptide in a composition is fused to a protease inhibitor and/or a therapeutic protein or therapeutic peptide.

In some aspects, the application relates to a nucleic acid (e.g., an expression construct, a plasmid, a vector, or a nucleic acid that is integrated in the genome of a cell) encoding a peptide or protein comprising a charge-complementary peptide and a protease inhibitor. In some aspects, the application relates to a nucleic acid (e.g., an expression construct, a plasmid, a vector, or a nucleic acid that is integrated in the genome of a cell) encoding a peptide or protein comprising a charge-complementary peptide, a protease inhibitor, and/or a therapeutic protein or therapeutic peptide. For example, in some embodiments, a nucleic acid (e.g., a nucleic acid comprising DNA, RNA, or a combination thereof) comprising a nucleotide sequence (e.g., a gene sequence) encoding a protein described herein (e.g., a protein comprising a charge-complementary peptide, a protease inhibitor, and/or a therapeutic protein or therapeutic peptide, such as wherein the charge-complementary peptide and the protease inhibitor, and/or the therapeutic protein or therapeutic peptide are connected directly or via a linker). In some embodiments, a nucleic acid comprises a vector (e.g., a plasmid).

In some aspects, the application relates to a cell (e.g., a bacterial cell or a mammalian cell, such as a human cell) or cell population thereof comprising a peptide or protein comprising a charge-complementary peptide and a protease inhibitor. In some embodiments, a cell or cell population comprises a nucleic acid encoding the peptide or protein. For example, in some aspects, the disclosure relates to a method comprising contacting a cell with a charge-complementary peptide comprising a protease inhibitor and/or a therapeutic protein or therapeutic peptide (e.g., a mammalian cell, such as a human cell), which includes, but is not limited to, an ex vivo or in vitro cell.

In some aspects, the application relates to compositions (e.g., pharmaceutical compositions, such as compositions comprising one or more peptides or proteins a pharmaceutically-acceptable carrier or buffer) and methods (e.g., methods comprising contacting a cell with one or more peptides or proteins and methods comprising administering one or more peptides or proteins, such as wherein the administration comprises subcutaneous administration, intraocular administration, intravitreal administration, subcutaneous administration, intravenous administration, intracerebroventricular administration, intramuscular administration, intracranial administration, intrathecal administration, intraperitoneal administration, direct injection to a target tissue or target organ, or any combination thereof). In some embodiments, hydrogel may be implanted directly at a target site, for example during a surgical procedure. In some embodiments, the compositions (e.g., hydrogels) are implanted subcutaneously, intraperitoneally, or at a disease site. In some embodiments, the compositions (e.g., pharmaceutical compositions, such as compositions comprising one or more peptides or proteins a pharmaceutically-acceptable carrier or buffer; or such as hydrogels) are implanted at a target site to form a depot, providing localized and sustained release of the therapeutic protein or therapeutic peptide. The depot may be formed subcutaneously, intraperitoneally, or directly at a disease site.

In some embodiments, a composition (e.g., a pharmaceutical composition that can be used for administration to a subject) comprises at least one peptide or protein comprising a charge-complementary peptide and a protease inhibitor (e.g., a positive peptide fused to a protease inhibitor and/or a negative peptide fused to a protease inhibitor) and at least one other peptide or protein (e.g., a therapeutic peptide or therapeutic protein). In some embodiments, the at least one other peptide or protein is fused to a charge-complementary peptide (e.g., wherein the at least one other peptide or protein comprises a peptide or protein fused to a positive peptide and/or a peptide or protein fused to a negative peptide, such as wherein the charge-complementary peptide is a CATCH peptide and/or the at least one or peptide or protein is a therapeutic peptide or protein). In some embodiments, a therapeutic peptide or therapeutic protein is capable of producing a physiological change that is associated with and/or expected to at least partially, if not fully, remedy at least one symptom associated with a disease, disorder, or condition (e.g., when it is administered in a therapeutically effective amount) in a subject (e.g., a subject having a genetic disease, a cancer, an inflammatory disease or an inflammatory condition, an autoimmune disease, a spleen disease, a lung disease, a hematological disease, a neurological disease, a painful condition, a psychiatric disorder, a metabolic disorder, an immune disorder, an infection of a pathogen, a kidney disease, a cardiovascular disease, a pancreatic disease, an intestinal disease, a retinal disease, a neuromuscular disease, a musculoskeletal disease, a lysosomal storage disease, or other disease, or any combination thereof). In some embodiments, the subject (e.g., human subject) has an inflammatory condition. In some embodiments, the subject (e.g., human subject) has an inflammatory condition and is administering a therapeutically effective amount of a peptide or protein comprising a charge-complementary peptide and a protease inhibitor, and further comprising adenosine synthase A (AdsA). In some embodiments, the subject (e.g., human subject) has an inflammatory condition and is administering a therapeutically effective amount of a peptide or protein comprising a charge-complementary peptide and leupeptin, and further comprising adenosine synthase A (AdsA). In some embodiments, the subject (e.g., human subject) has an inflammatory condition and is administering a therapeutically effective amount of a peptide or protein comprising a charge-complementary peptide and oxidized leupeptin, and further comprising adenosine synthase A (AdsA). In some embodiments, the subject (e.g., human subject) has an inflammatory condition and is administering a therapeutically effective amount of a peptide or protein comprising a charge-complementary peptide and reduced leupeptin, and further comprising adenosine synthase A (AdsA).

In some embodiments, a composition (e.g., a pharmaceutical composition that can be used for administration to a subject) comprises one or more peptides or proteins at a concentration of 1 ng-1,000 mg (e.g., 1 ng-100 ng, 100 ng-1,000 ng, 1 μg-100 μg, 100 μg-1,000 μg, 1 mg-10 mg, 10 mg-25 mg, 25 mg-50 mg, 50 mg-100 mg, 100 mg-250 mg, 250 mg-500 mg, or 500 mg-1,000 mg).

As a further example, in some aspects the application relates to a method comprising administering a charge-complementary peptide comprising a protease inhibitor and/or a therapeutic protein or therapeutic peptide to a subject (e.g., a mammalian subject, such as a human subject), such as wherein the method comprises administering a composition (e.g., a pharmaceutical composition) to a subject in need thereof. In some embodiments, the subject is characterized as having or at risk of having a disease, disorder, or condition (e.g., wherein therapeutic protein or therapeutic peptide is selected to treat the disease, disorder, or condition). In some aspects, the disclosure provides methods of reducing an anti-drug antibody (humoral) response to the therapeutic protein or therapeutic peptide. In some embodiments, administration of a charge-complementary peptide comprising a protease inhibitor (e.g., wherein the charge-complementary peptide comprising the protease inhibitor is provided in a composition, such as a pharmaceutical composition) to a subject is performed subcutaneously, subcutaneously, intravenously, intramuscularly, or by direct injection to one or more cells, tissues, or organs. In some embodiments, administration of a charge-complementary peptide comprising a protease inhibitor (e.g., wherein the charge-complementary peptide comprising the protease inhibitor is provided in a composition, such as a pharmaceutical composition) is performed a plurality of times (e.g., two, three, four, or more than four times), for example, the administration is performed repeatedly. In some embodiments, the disclosure provides methods of administering the charge-complementary peptides fused to a protease inhibitor and a therapeutic protein or therapeutic peptide (e.g., as a hydrogel or as a composition) and further comprises administering the therapeutic protein or therapeutic peptide repeatedly (e.g., 1, 2, 3, 4, 5 or more times) to the subject (e.g., without readministering the hydrogel or composition). In some embodiments, administration of a charge-complementary peptide comprising a protease inhibitor and a therapeutic protein or therapeutic peptide (e.g., wherein the charge-complementary peptide comprising the protease inhibitor is provided in a composition, such as a pharmaceutical composition) results in a reduced level of anti-drug antibodies relative to that of a control. In some embodiments, a control is a similarly situated subject who has not been administered the charge-complementary peptide comprising a protease inhibitor and a therapeutic protein or therapeutic peptide (e.g., wherein the charge-complementary peptide comprising the protease inhibitor is provided in a composition, such as a pharmaceutical composition). Anti-drug antibodies can be measured by any method known in the art (for example, using bridging electrochemiluminescence (ECL) immunoassays). In some embodiments, anti-drug antibodies are reduced 1.5-fold, 2-fold, 2.5-fold, 3-fold, 3.5-fold, 4-fold, or more.

In some embodiments, a method comprises contacting charge-complementary peptides, including at least one positively charged peptide and at least one negatively charged peptide, thereby forming a supramolecular structure (e.g., a particle, a granule, or a hydrogel), wherein one or more of the charge-complementary peptides are fused to a protease inhibitor and, optionally wherein one or more of the charge-complementary peptides are fused to a therapeutic protein or therapeutic peptide. In some embodiments, charge-complementary peptides, wherein one or both of the charge-complementary peptides comprise a protease inhibitor, are mixed under stimulating conditions that promote co-assembly of the charge-complementary peptides in a supramolecular structure (e.g., a granule, a particle, and/or a hydrogel).

In some embodiments, conditions that promote co-assembly of charge-complementary peptides (e.g., co-assembly of charge-complementary peptides in a composition, such as a pharmaceutical composition) involve a crowding agent (e.g., polyethylene glycol (PEG)), providing the charge-complementary peptides in an aqueous solution (e.g., a neutral aqueous solution), and/or providing the charge-complementary peptides in a non-equal molar ratio of positively charged peptide to negatively charged peptide (see, e.g., the description of stimulating conditions in International Patent Application No. PCT/US2022/025426 (published as WO 2022/225,987) and International Patent Application No. PCT/US2023/065823 (published as WO 2023/201,370)), which are each incorporated by reference in their entirety, including their description of charge-complementary peptides and conditions that promote co-assembly of charge-complementary peptides and crowding agents that promote co-assembly of charge-complementary peptides.

For example, in some embodiments, the molarity of the positively charged peptide is greater than the molarity of the negatively charged peptide in a solution (e.g., wherein the solution is a composition, such as a pharmaceutical composition). In some embodiments, the non-equal molar ratio of negatively charged peptide to positively charged peptide in the solution is 1:2, 1:50, or any intervening ratio thereof, such as 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, or 1:49. In some embodiments, the non-equal molar ratio of negatively charged peptide to positively charged peptide in the solution is between 1:15 and 1:30. In some embodiments, the non-equal molar ratio of negatively charged peptide to positively charged peptide in the solution is 1:23.

In some embodiments, the molarity of the negatively charged peptide is greater than the molarity of the positively charged peptide in a solution (e.g., wherein the solution is a composition, such as a pharmaceutical composition). In some embodiments, the non-equal molar ratio of positively charged peptide to negatively charged peptide in the solution is 1:2, 1:50, or any intervening ratio thereof, such as 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, or 1:49.

In some embodiments, conditions that promote co-assembly of charge-complementary peptides (e.g., co-assembly of charge-complementary peptides in a pharmaceutical composition) involve contacting charge-complementary peptides (e.g., a positive peptide and a negative peptide that are in an aqueous solution, which includes, but is not limited to, solutions wherein the positive and negative peptides are in non-equal molar ratios) with one or more crowding agents (e.g., 1, 2, 3, 4, 5, or more than 5 crowding agents). Non-limiting examples of crowding agents include sucrose, glycerol, Polysorbate 20 (IUPAC: Polyoxyethylene (20) sorbitan monolaurate; commercially known as “Tween 20”™), polysorbate 80 (IUPAC: Polyoxyethylene (20) sorbitan monooleate; commercially known as “Tween 80”™), (1,1,3,3-Tetramethylbutyl)phenyl-polyethylene glycol, Polyethylene glycol tert-octylphenyl ether (commercially known as “Triton X-114”™), sodium dodecylsulfate (SDS), deoxycholate sodium, (3-((3-cholamidopropyl)dimethylammonio)-1-propanesulfonate) (commonly known as CHAPS detergent), benzalkonium chloride, polyethylene glycol 1000 (PEG 1000), PEG 1500, PEG 1550, PEG 2000, PEG 3350, PEG 6000, PEG 20000, PEG 50000, Ficoll 70, Ficoll 400, serum albumin, and dextran (50k or 500k). In some embodiments, the one or more crowding agents comprise polyethylene glycol. In some embodiments, the one or more crowding agents comprise PEG 1500. In some embodiments, the one or more crowding agents are provided in a concentration of at least 0.1 μg/mL (e.g., at least 1 μg/mL, at least 10 μg/mL, at least 100 μg/mL, at least 250 μg/mL, or at least 500 μg/mL). In some embodiments, the one or more crowding agents are provided in a concentration of 0.1 μg/mL-1,000 μg/mL (e.g., 0.1 μg/mL-1 μg/mL, 1 μg/mL-10 μg/mL, 10 μg/mL-100 μg/mL, 100 μg/mL-200 μg/mL, 200 μg/mL-300 μg/mL, 300 μg/mL-400 μg/mL, 400 μg/mL-500 μg/mL, 500 μg/mL-600 μg/mL, 600 μg/mL-700 μg/mL, 700 μg/mL-800 μg/mL, 800 μg/mL-900 μg/mL, 900 μg/mL-1,000 μg/mL). In some embodiments, the one or more crowding agents are provided in a concentration of 0.1 μg/mL-100 μg/mL (e.g., 0.1 μg/mL-1 μg/mL, 1 μg/mL-10 μg/mL, 10 μg/mL-20 μg/mL, 20 μg/mL-30 μg/mL, 30 μg/mL-40 μg/mL, 40 μg/mL-50 μg/mL, 50 μg/mL-60 μg/mL, 60 μg/mL-70 μg/mL, 70 μg/mL-80 μg/mL, 80 μg/mL-90 μg/mL, 90 μg/mL-100 μg/mL). In some embodiments, the one or more crowding agents are provided in a concentration of 10 μg/mL-100 μg/mL.

In some embodiments, a composition (e.g., a pharmaceutical composition) comprises an assembly (e.g., a supramolecular structure, such as a particle, a granule, or a hydrogel) of co-assembling peptides (e.g., charge-complementary peptides), wherein at least one of the co-assembling peptides comprise a protease inhibitor and wherein the assembly was formed in the presence of one or more crowding agents. In some embodiments, a composition that comprises an assembly of co-assembling peptides (e.g., a composition for administration to a subject, such as a pharmaceutical composition) and one or more crowding agents that were used to form the assembly. In some embodiments, the concentration of one or more crowding agents that were used to form an assembly of co-assembling peptides in a composition (e.g., a composition for administration to a subject, such as a pharmaceutical composition) are adjusted (e.g., increased or decreased) following formation of the assembly. For example, in some embodiments, the concentration of the one or more crowding agents in the composition are increased or decreased by at least 1.1-fold (e.g., increased or decreased by 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 4.0-fold, 5.0-fold, 6.0-fold, 7.0-fold, 8.0-fold, 9.0-fold, 10.0-fold, or more than 10.0-fold) following co-assembly of charge-complementary peptides (e.g., wherein increasing or decreasing the concentration of the one or more crowding agents in the composition comprises using dialysis, gel filtration chromatography, ultrafiltration, precipitation and re-dissolution, lyophilization and reconstitution, or a combination thereof). In some embodiments, one or more crowding agents are removed from a composition (e.g., a composition for administration to a subject, such as a pharmaceutical composition) comprising an assembly of co-assembling peptides, wherein the assembly was formed in the presence of the one or more crowding agents (e.g., wherein the one or more crowding agents were removed from the composition using dialysis, gel filtration chromatography, ultrafiltration, precipitation and re-dissolution, lyophilization and reconstitution, or a combination thereof).

In some embodiments, conditions that promote co-assembly of charge-complementary peptides (e.g., co-assembly of positively charged peptides and negatively charged peptides in a composition, such as a pharmaceutical composition) involve incubating the charge-complementary peptides in an aqueous solution having a pH of about 5.0 to about 8.5 (e.g., a pH of 5.0-5.5, 5.5-6.0, 6.0-6.5, 6.5-7.0, 7.0-7.5, 7.5-8.0, or 8.0-8.5). In some embodiments, the aqueous solution has a pH of about 6.0 to about 8.0 (e.g., a pH of 6.0-7.0 or 7.0-8.0). In some embodiments, the aqueous solution has a pH of about 6.5 to about 7.5 (e.g., a pH of 6.5, 6.6, 6.7, 6.8, 6.9, 7.0, 7.1, 7.2, 7.3, 7.4, or 7.5). In some embodiments, the aqueous solution is a neutral aqueous solution.

In some aspects, the application relates to a kit comprising one or more compositions (e.g., a pharmaceutical composition) that can be used to perform a method described herein. In some embodiments, a kit described herein includes one or more containers housing components of a composition for performing a method described herein. In some embodiments, the components may be prepared sterilely and shipped refrigerated or on ice. In some embodiments, a kit includes other components that can be used in a method described herein, such as syringes or vials comprising a pharmaceutical composition and/or instructions for using a composition described herein. A kit described herein can comprise components provided in liquid form (e.g., in solution) and/or components in solid form (e.g., a dry powder). In some embodiments, some of the components can be reconstituted or otherwise processed (e.g., processed into an active form), for example, by the addition of a suitable solvent (e.g., water), which may or may not be provided with the kit.

### EXAMPLES

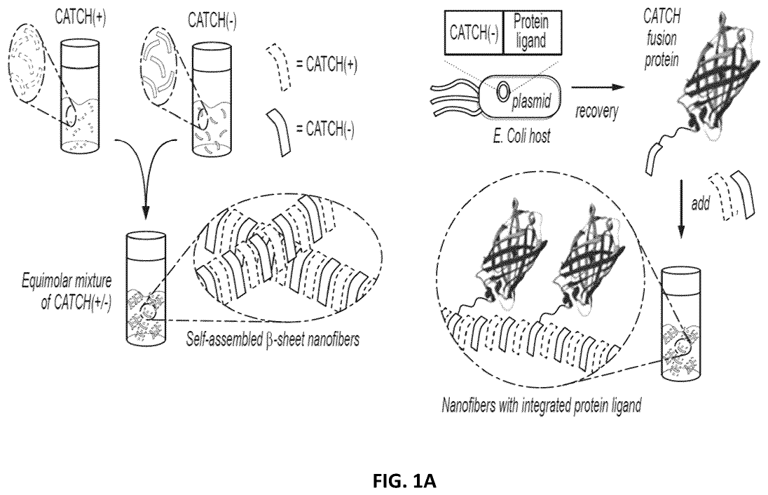

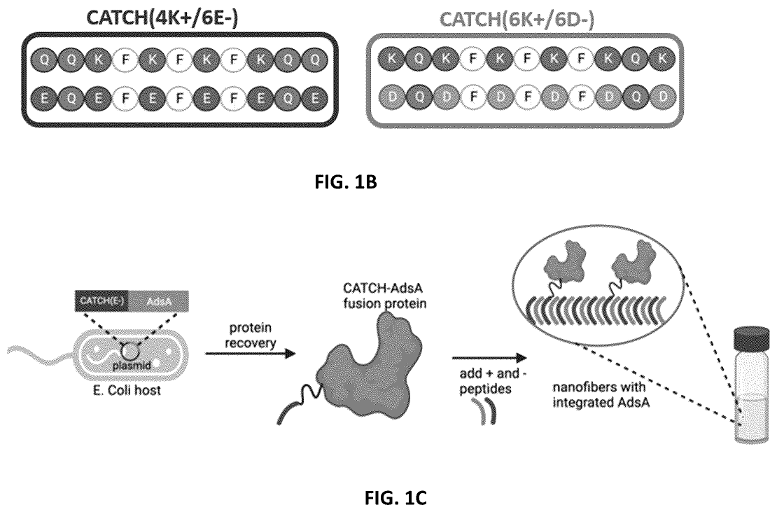

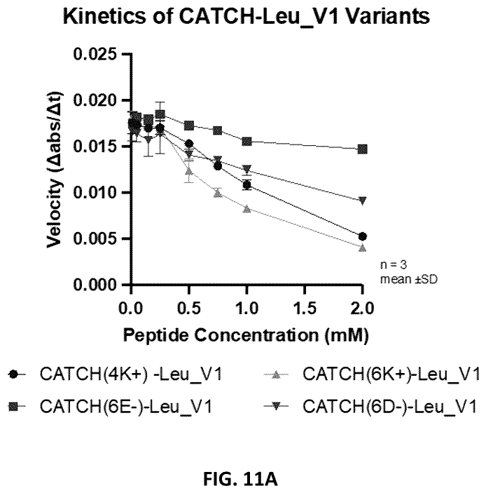

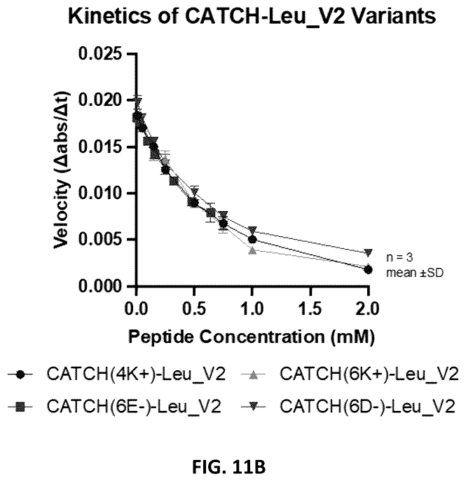

Non-limiting aspects of the application are described in FIGS. 1-12B, which show results obtained from CATCH-leupeptin immunogenicity studies. FIGS. 1A-1C are schematics illustrating the structure of co-assembling peptides based on charge complementarity (CATCH) proteins and their assembly into a vehicle for transporting therapeutic proteins (in this case, adenosine synthase A, AdsA).

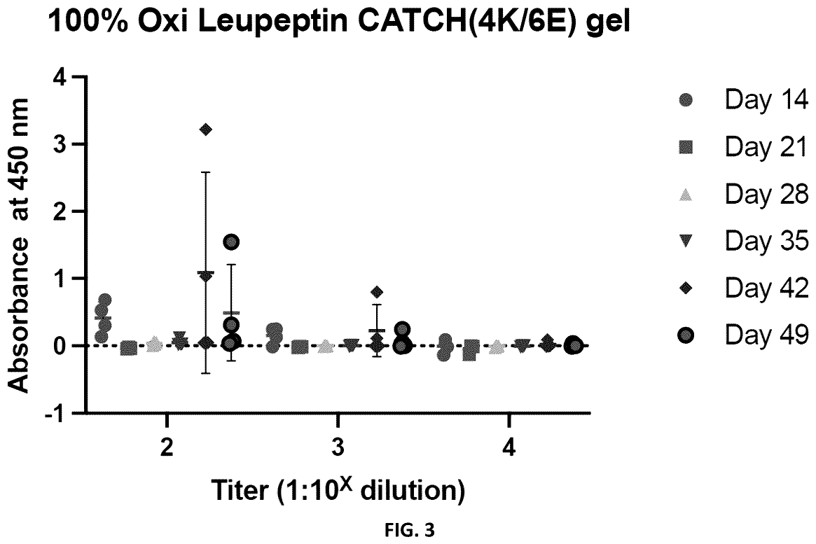

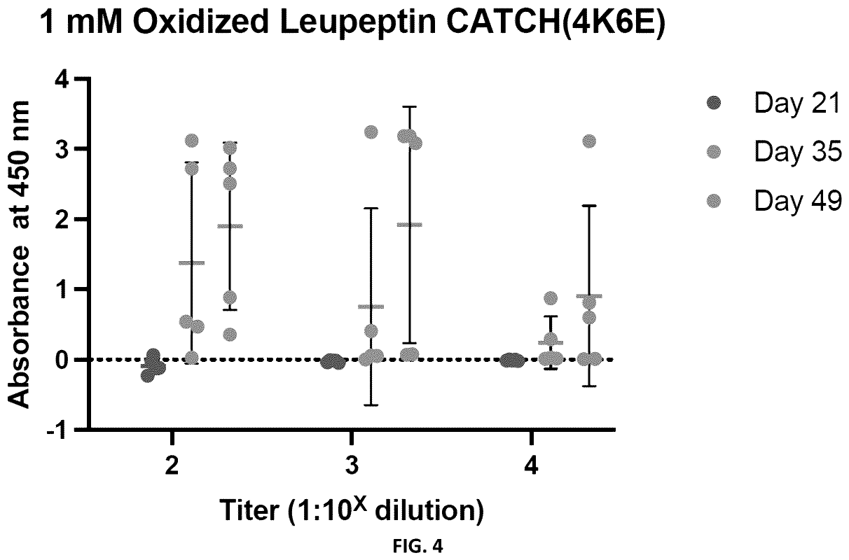

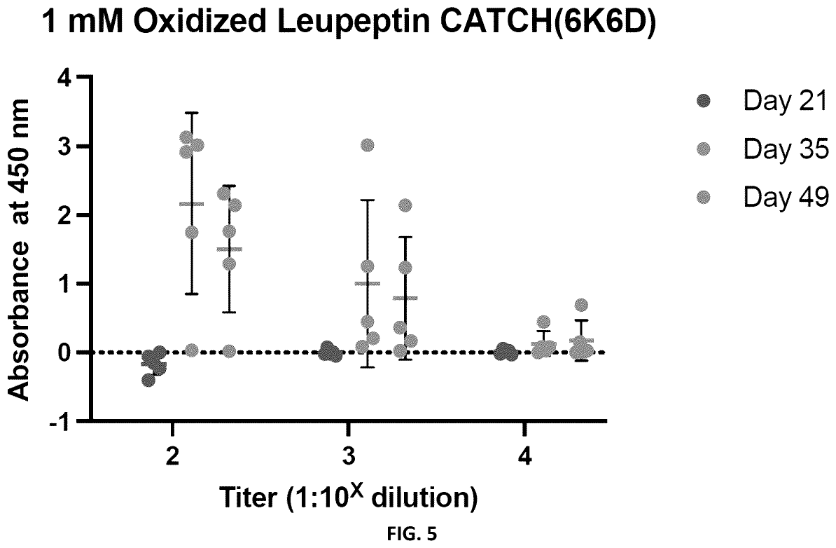

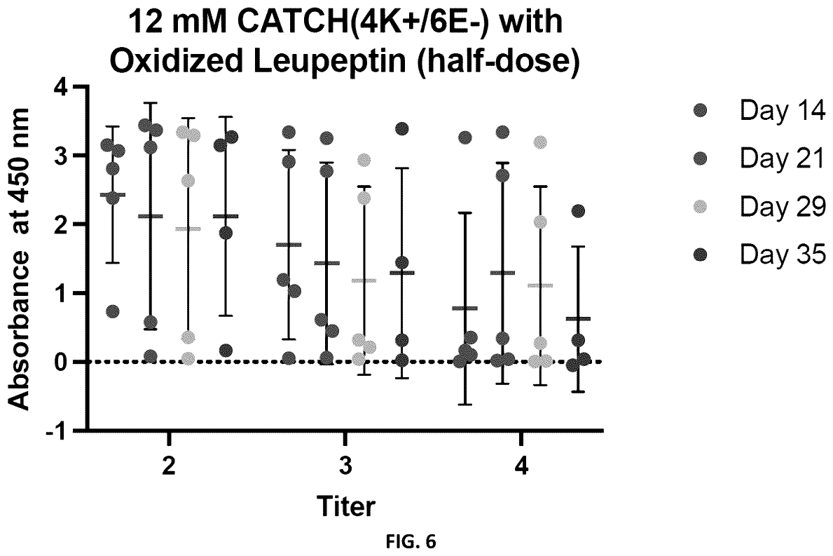

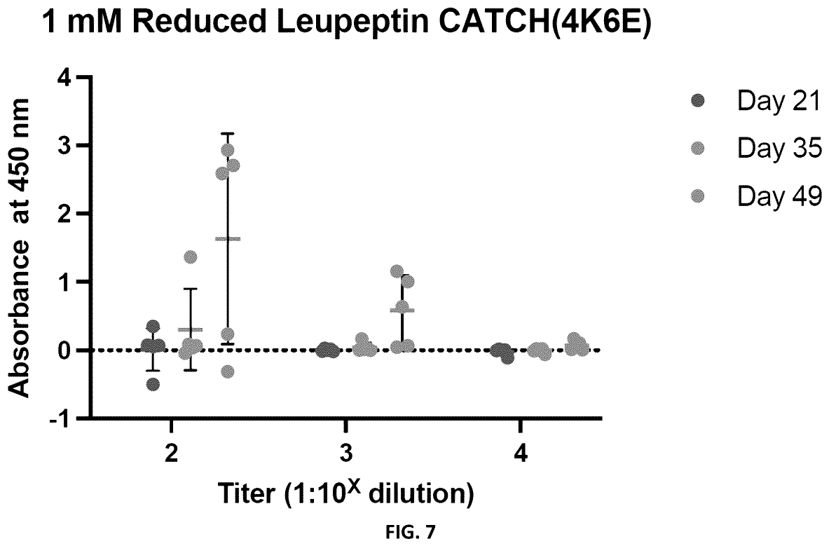

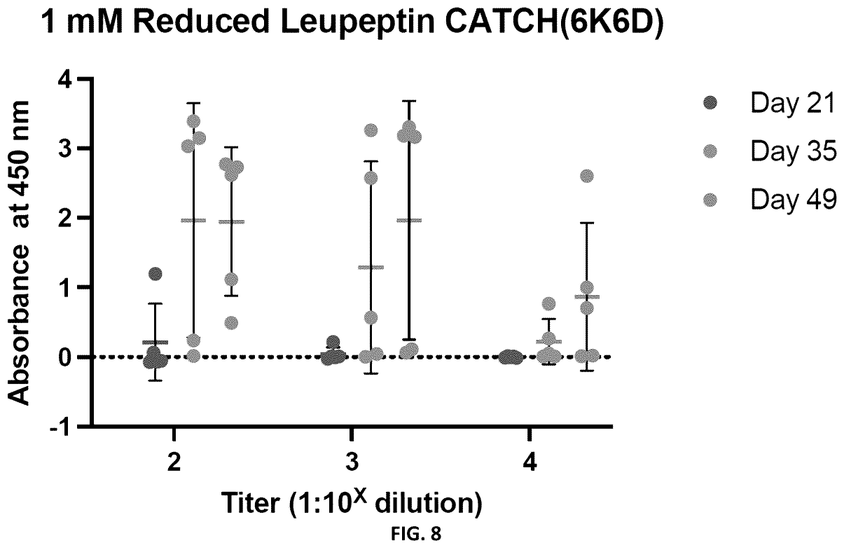

First, the effect of CATCH-leupeptin hydrogels containing different concentrations of leupeptin were evaluated. Briefly, 12 mM CATCH-Leupeptin hydrogels with CATCH (6E−)-AdsA (adenosine synthase A) were subcutaneously injected into the scruff of mice on day 0 and then boosted on day 28. Sera was collected weekly and evaluated for anti-AdsA antibodies using an ELISA to measure total IgG levels. In the group administered hydrogels containing 100% leupeptin (oxidized form), IgG responses were found to be minimal (FIG. 3). With lower doses of the hydrogel (1 mM), IgG responses were observed to increase over the three time points studied in both the 4K+/6E− (FIG. 4) and 6K+/6D− (FIG. 5) configurations. At lower concentrations of oxidized leupeptin (50% dose), IgG responses were observed to be increased relative to those of the full dose (100%) (FIG. 6). Using reduced leupeptin at a lower dose of the hydrogel (1 mM), it was observed that the titers were lower compared to those from lower concentrations of oxidized leupeptin (50% dose) in both the 4K+/6E− (FIG. 7) and 6K+/6D− (FIG. 8) configurations.

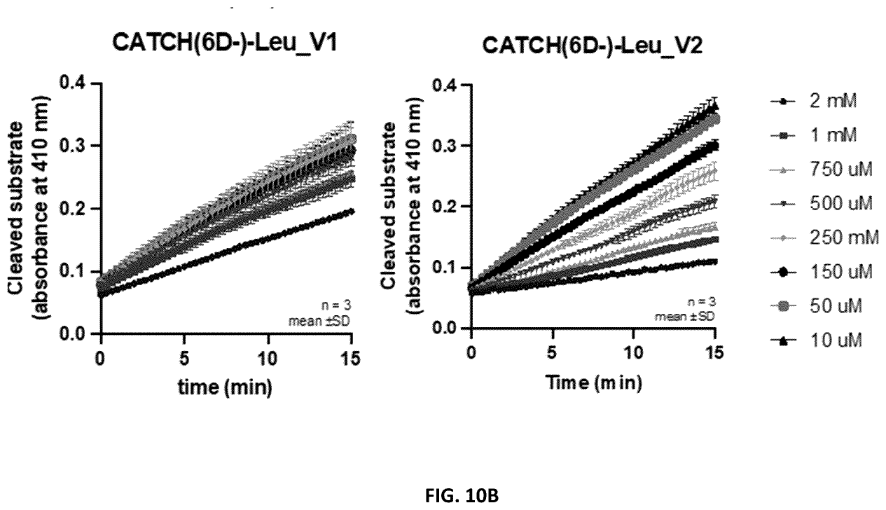

Next, IC₅₀ values for different CATCH-leupeptin variants were determined. The results, shown in FIG. 2 and measured by an inhibition assay, indicated that the inhibitory properties of leupeptin are preserved in this form. In general, the reduced form of leupeptin seemed to perform better than the oxidated form. For example, cleavage activity (indicating protease activity) was screened (FIGS. 10A-10B). As shown in FIGS. 10A-10B, the reduced form of leupeptin was more active (had more protease activity). Similarly, a kinetic analysis, looking at the rate of protease inhibition over the time for both the oxidized and reduced forms of leupeptin demonstrated that the reduced form had better inhibitor activity.

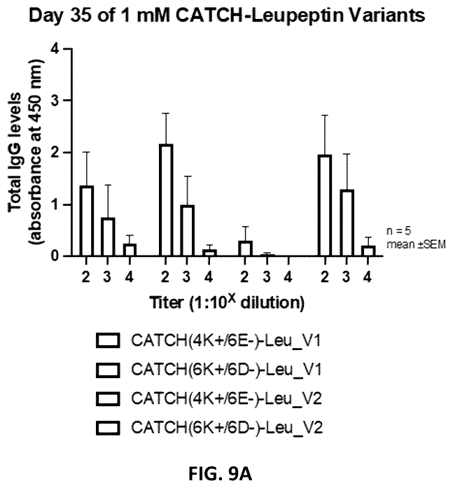

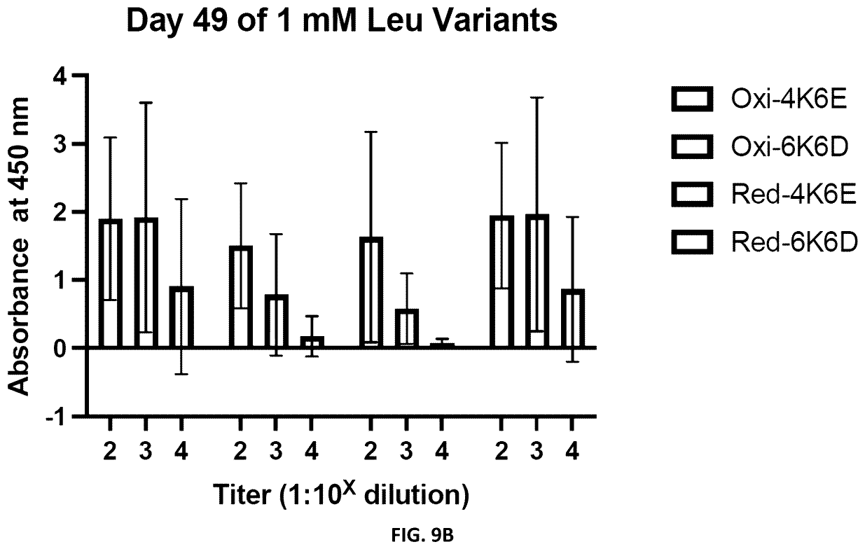

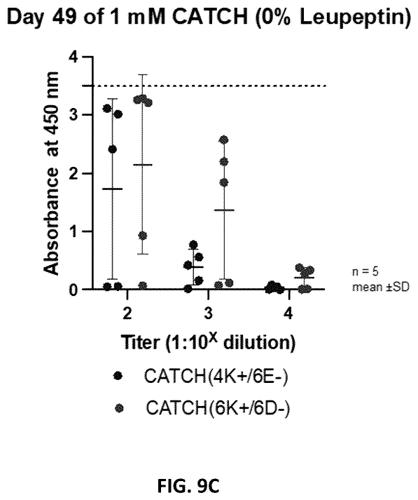

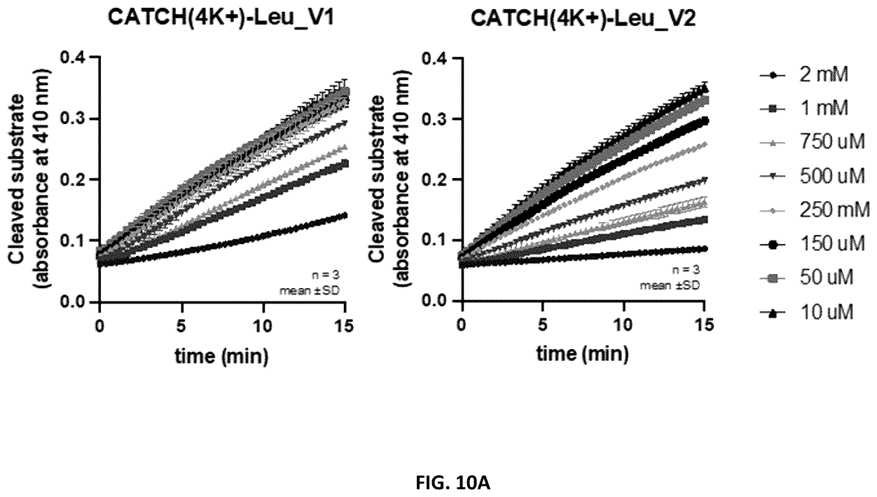

Next, IgG levels in mice after administration of CATCH-leupeptin at sub-hydrogel concentrations was examined using the same protocol as above (two doses, separated by four weeks and weekly sampling). Each CATCH-leupeptin variant was tested at a total peptide concentration of 1 mM. The results are shown in FIGS. 9A-9C, and demonstrate that, relative to each other, CATCH (4K+/6E−)-Leu_V2 (reduced form) injections resulted in the lowest anti-AdsA IgG titers by day 49 of the study. The results indicated that IgG levels are decreased when a protein was co-administered with a CATCH peptide fused to leupeptin (see, e.g., the day 49 results observed with Oxi-6K6D and Red-4K6E, as presented in FIG. 9B).

Solid phase peptide synthesis was used to synthesize CATCH peptides bearing the leupeptin sequence. Based on KI values for leupeptin inhibition on trypsin, two forms of CATCH-Leupeptin were used: a reduced (CH₂OH) and an oxidized (COOH) form (relative to the natural aldehyde). Four CATCH peptides, CATCH (4K+), CATCH (6K+), CATCH (6E−), and CATCH (6D−) were synthesized with either leupeptin variant, and then admixed with their charge-complementary partner to form fibrils (4K+/6E− or 6K+/6D−). A fusion of CATCH and Adenosine synthase A (AdsA) was co-assembled into the fibers as the therapeutic protein. CATCH-leupeptin/CATCH-AdsA gels were administered on Day 0 and Day 28, with weekly blood draws (n=5 per treatment group). ELISA was used to quantify total IgG raised against AdsA.

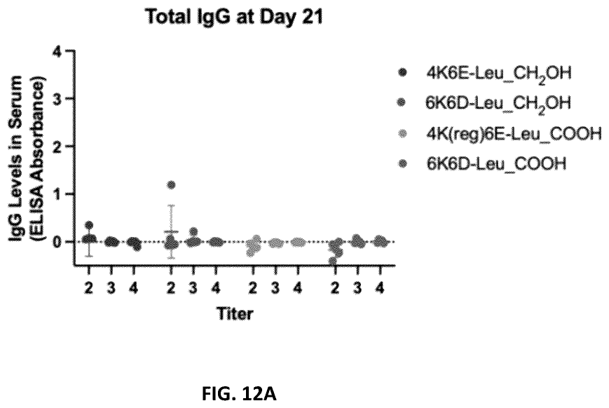

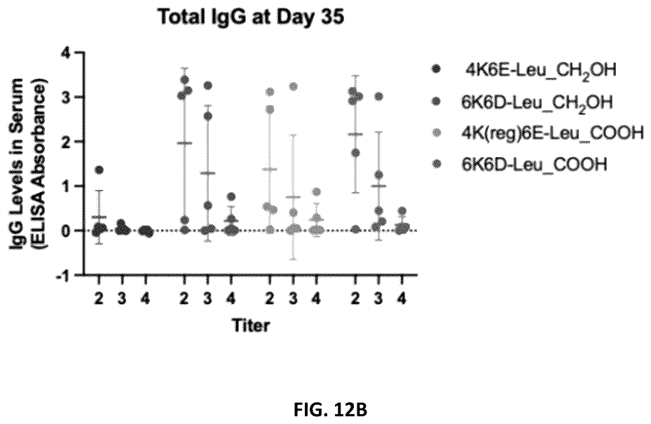

Four CATCH peptide pairs with two different leupeptin chemistries were evaluated for their ability to reduce immunogenicity of AdsA. FIGS. 12A-12B show the total IgG in serum versus serum dilution (titer 2−4=10²-10₄ dilution), at days 21 and 35. FIG. 12A demonstrates that by day 21, no measurable antibodies were raised against AdsA. By day 35, groups that received either of the CATCH (6K+/6D−)-leupeptin variants had positive anti-AdsA titers. In contrast, only one animal was positive at the lowest titer in the group that received CATCH (4K+/6E−)-LeuCH₂OH, suggesting that this material system inhibited anti-AdsA antibody emergence. Consistent with this, the group that received materials made from unmodified CATCH (4K+) and CATCH (6E−)-LeuCOOH induced a response that was similar to the CATCH (6K+/6D−)-Leu groups. It was previously found that mechanics and architecture play a role in the adjuvanticity of the CATCH materials, which is consistent with these modified versions; however, compared to the parent peptide their architectures appear altered, as evaluated by ThT fluorescence microscopy (data not shown). The effectiveness of CATCH (4K+/6E−)-LeuCH₂OH was somewhat unexpected because the oxidized form (COOH) was reported to have a hundred-fold better inhibitory constant than the reduced form (2.7 μM vs 270 μM). This suggests that conjugation of the leupeptin sequence onto CATCH peptide may affect leupeptin inhibitory properties.

Accordingly, the CATCH peptide platform allows for the delivery of enzyme therapeutics directly to a disease site. Although promising, immunogenicity remains a concern. Introducing the leupeptin sequence onto the CATCH peptides is one approach to engineer the material itself to block antigen processing of the enzyme cargo.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A peptide or protein comprising a charge-complementary peptide and a protease inhibitor.

2. The peptide or protein of claim 1, wherein the charge-complementary peptide comprises a positive peptide comprising SEQ ID NO: 1 or 3.

3. The peptide or protein of claim 1, wherein the charge-complementary peptide comprises a negative peptide comprising SEQ ID NO: 2 or 4.

4. The peptide or protein of claim 1, wherein the protease inhibitor comprises a leupeptin or a modified form thereof.

5. The peptide or protein of claim 1, wherein the protease inhibitor comprises a leupeptin having the sequence LLR.

6. The peptide or protein of claim 5, wherein the leupeptin is in an oxidized form.

7. The peptide or protein of claim 5, wherein the leupeptin is in a reduced form.

8. The peptide or protein of claim 1, wherein the protease inhibitor comprises a Pepstatin A or a modified form thereof.

9. The peptide or protein of claim 1, wherein the protease inhibitor comprises an amino acid sequence of {Iso-Val} {Val} {Val} {Sta} {Ala} {Sta}, wherein “Iso-Val” is isovaline and “Sta” is statine (SEQ ID NO: 18).

10. The peptide or protein of claim 1, further comprising a therapeutic protein or therapeutic peptide.

11. (canceled)

12. The peptide or protein of claim 10, wherein the therapeutic protein or therapeutic peptide is linked to the peptide or protein via a glycine-serine rich linker.

13.-14. (canceled)

15. A composition comprising a plurality of positive peptides, a plurality of negative peptides, and a protease inhibitor fused to one or more of the plurality of positive peptides, or to one or more of the plurality of negative peptides, or to one or more of the plurality of positive peptides and one or more of the plurality of negative peptides, wherein the positive peptides and negative peptides are co-assembling peptides.

16. The composition of claim 15, wherein the plurality of positive peptides comprises SEQ ID NO: 1 or 3.

17. The composition of claim 15, wherein the plurality of negative peptides comprises SEQ ID NO: 2 or 4.

18. (canceled)

19. The composition of claim 15, wherein the protease inhibitor comprises a leupeptin having the sequence LLR.

20. The composition of claim 19, wherein the leupeptin is in an oxidized form.

21. The composition of claim 19, wherein the leupeptin is in a reduced form.

22. The composition of claim 15, wherein the protease inhibitor comprises a Pepstatin A or a modified form thereof.

23. The composition of claim 15, wherein the protease inhibitor comprises an amino acid sequence of {Iso-Val} {Val} {Val} {Sta} {Ala} {Sta}, wherein “Iso-Val” is isovaline and “Sta” is statine (SEQ ID NO: 18).

24. The composition of claim 15, further comprising
a therapeutic protein or therapeutic peptide linked to one or more of the plurality of positive peptides, or to one or more of the plurality of negative peptides, or to one or more of the plurality of positive peptides and one or more of the plurality of negative peptides, via a linker.

25-34. (canceled)
